(19) United States
(12) Patent Application Publication
(10) Pub. No.: US 2008/0039775 A1
(43) Pub. Date: Feb. 14, 2008

(54) Apparatus and Method for Transdermal Delivery of Parathyroid Hormone Agents to Prevent or Treat Osteopenia

(75) Inventors: Mahmoud Ameri, Fremont, CA (US); Michel J.N. Cormier, Mountain View, CA (US); Yuh-Fun Maa, Millbrae, CA (US); Marika Kamberi, San Jose, CA (US); Peter Daddona, Menlo Park, CA (US)

Correspondence Address: WILSON SONSINI GOODRICH & ROSATI, 650 PAGE MILL ROAD, PALO ALTO, CA, 94304-1050, US

(73) Assignee: ALZA CORPORATION, Mountain View, CA (US)

(21) Appl. No.: 11/686,909

(22) Filed: Mar. 15, 2007

Related U.S. Application Data
(60) Provisional application No. 60/782,939, filed on Mar. 15, 2006.

(51) Int. Cl. A61B 17/20

(52) U.S. Cl. 604/46

(57) ABSTRACT

An apparatus and method for transdermally delivering a biologically active agent to prevent or treat osteopenia, comprising a delivery system having a microprojection member (or system) that includes a plurality of microprojections (or array thereof) that are adapted to pierce through the stratum corneum into the underlying epidermis layer, or epidermis and dermis layers. In one embodiment, the PTH-based agent is contained in a biocompatible coating that is applied to the microprojection member.

### CROSS-REFERENCE

This application claims the benefit of U.S. Provisional Application No. 60/782,939, filed Mar. 15, 2006, the content of which is incorporated herein by reference in their entirety.

### FIELD OF THE PRESENT INVENTION

The present invention relates generally to methods of using transdermal agent delivery systems. More particularly, the invention relates to a method for transdermal delivery of parathyroid hormone agents to patients to prevent or treat osteopenia.

### BACKGROUND OF THE INVENTION

Active agents (or drugs) are most conventionally administered either orally or by injection. Unfortunately, many active agent are completely ineffective or have radically reduced efficacy when orally administered, since they either are not absorbed or are adversely affected before entering the bloodstream and thus do not possess the desired activity. On the other hand, the direct injection of the agent intravenously or subcutaneously, while assuring no modification of the agent during administration, is a difficult, inconvenient, painful and uncomfortable procedure that sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and devices developed to mechanically penetrate or disrupt the outermost skin layers thereby creating pathways into the skin in order to enhance the amount of agent being transdermally delivered. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is possible to have the active agent that is to be delivered coated on the microprojections instead of contained in a physical reservoir. This eliminates the necessity of a separate physical reservoir and developing an agent formulation or composition specifically for the reservoir.

As is well known in the art, osteoporosis is a bone disorder characterized by progressive bone loss that predisposes an individual to an increased risk of fracture, typically in the hip, spine and wrist. The progressive bone loss, which typically begins between the ages of 30 and 40, is mainly asymptomatic until a bone fracture occurs, leading to a high degree of patient morbidity and mortality. Eighty percent of those affected by osteoporosis are women and, based on recent studies, during the six years following the onset of menopause, women lose one third of their bone mass.

As is also well known in the art, parathyroid hormone (PTH) is a hormone secreted by the parathyroid gland that regulates the metabolism of calcium and phosphate in the body. PTH has stirred great interest in the treatment of osteoporosis for its ability to promote bone formation and, hence, dramatically reduced incidence of fractures. Large-scale clinical trials have shown that PTH effectively and safely reduces the percentage of vertebral and non-vertebral fractures in women with osteoporosis.

PTH-based agents have also stirred interest in the treatment of bone fractures (in both men and women) by virtue of their ability to accelerate bone healing.

To this end, various stabilized formulations of PTH-based agents have been developed that can be reconstituted for subcutaneous injection, which, as discussed below, is the conventional means of delivery. Illustrative are the formulations disclosed in U.S. Pat. No. 5,563,122 (“Stabilized Parathyroid Hormone Composition”) and U.S. Pat. No. 7,144,861 (“Stabilized Teriparatide Solutions”), which are incorporated by reference herein in their entirety.

A currently approved injectable PTH-based agent is FORTEO™ (an rDNA derived teriparatide injection), which contains recombinant human parathyroid hormone (1-34), (rhPTH (1-34)). FORTEO™ is typically prescribed for women with a history of osteoporotic fracture, who have multiple risk factors for fracture, or who have failed or are intolerant of previous osteoporosis therapy, based on a physician's assessment. In postmenopausal women with osteoporosis, FORTEO™ has been found to increase bone mineral density (BMD) and reduce the risk of vertebral and non-vertebral fractures.

FORTEO™ has also been found to increase bone mass in men with primary or hypogonadal osteoporosis who are at high risk for fracture. These include men with a history of osteoporotic fracture, or who have multiple risk factors for fracture, or who have failed or are intolerant to previous osteoporosis therapy. In men with primary or hypogonadal osteoporosis, FORTEO™ has similarly been found to increase BMD.

In addition to subcutaneous injection, other means of delivering PTH-based agents have also been investigated. For example, various pulmonary delivery (i.e., inhalation) methods are discussed in “Pulmonary Delivery of Drugs for Bone Disorders,” Advanced Drug Delivery Reviews, Vol. 42, Issue 3, pp. 239-248 (Aug. 31, 2000), Patton, “Bioavailability of Pulmonary Delivered Peptides and Proteins: -Interferon, Calcitonins and Parathyroid Hormones,” Journal of Controlled_{—}Release, Vol.28, Issues 1-3, pp. 79-85 (January 1994), Patton, et al., “Impact of Formulation and Methods of Pulmonary Delivery on Absorption of Parathyroid Hormone (1-34).from Rat Lungs,” Journal of Pharmaceutical Sciences, Vol. 93, Issue 5, pp. 1241-1252 (May 2004), Codrons, et al., “Systemic Delivery of Parathyroid Hormone (1-34) Using Inhalation Dry Powders in Rats,” Journal of Pharmaceutical_{—}Sciences, Vol. 92, Issue 5, pp. 938-950 (May 2003) and Pfützner, A, et al., “Pilot Study with Technosphere/PTH(1-34)—A New Approach for Effective Pulmonary Delivery of Parathyroid Hormone (1-34)”, Horm. Metab. Res., Vol.35(5), pp. 319-23.

Various methods of active transdermal delivery of PTH-based agents are also discussed in “The Effect of Electroporation on Eontophoretic Eransdermal Delivery of Calcium Regulating Hormones,” Journal of Controlled Release, Vol. 66, Issues 2-3, pp. 127-133 (May 15, 2000) and Chang, et al., “Prevention of Bone Loss in Ovariectomized Rats by Pulsatile Transdermal Iontophoretic Administration of Human PTH(1-34),” Journal of Pharmaceutical Sciences, Vol. 91, Issue 2, pp.350-361 (February 2002).

Despite the efficacy of PTH in treating disorders such as osteoporosis, there are several drawbacks and disadvantages associated with the disclosed prior art methods of delivering PTH, particularly, via subcutaneous injection. A major drawback is that subcutaneous injection is a difficult and uncomfortable procedure, which often results in poor patient compliance.

Intracutaneous administration of agents, such as hGH, using microprojection systems has previously been documented to provide a pharmacokinetic profile of hGH similar to that observed following subcutaneous administration. See, e.g., Cormier, et al., U.S. Patent Application Pub. No. 2002/0128599, entitled “Transdermal Drug Delivery Devices Having Coated Microprotrusions”.

Continuous infusion of a PTH-based agent in vivo results in active bone resorption. It is therefore of critical importance that the PTH-based agent be administered in a pulsatile fashion. Based on the efficacy results from the once daily subcutaneous injection, any alternative route of PTH delivery should provide blood concentration of PTH no slower than that for subcutaneously injected PTH.

A solution to some of the remaining problems represented by current PTH-based delivery systems was disclosed in WO/2005/112984, wherein an apparatus and method were identified to deliver PTH-based agents. The apparatus and method comprised a delivery system having a microprojection member (or system) that included a plurality of microprojections (or array thereof) that were adapted to pierce through the stratum corneum into the underlying epidermis layer, or epidermis and dermis layers and allow the delivery of PTH-based agents. In one embodiment, methods were identified for using the delivery system to treat osteoporosis and osteoporotic fractures. While the use of the delivery system identified in WO/2005/112984 is a significant advance to treat osteoporosis and osteoporotic fractures, it would be better if the osteoporosis and osteoporotic fractures had never occurred.

Osteopenia is a medical condition that refers to decreased calcification or density of bone. Having osteopenia places a person at risk for developing osteoporosis and the difference between the two conditions is generally described in terms of bone density. For example, bone density can be described in relationship to what it should be in young women; it is expressed as a standard deviation from the mean (average) bone density in a 35-year-old. Within 1 standard deviation of the mean in either direction is considered normal. A bone density within the range of 1 to 2.5 standard deviations below the mean is defined as osteopenia, and greater than 2.5 standard deviations below the mean is osteoporosis. If one were to successfully treat an individual with osteopenia, then it can be reasonably argued that there is a significant likelihood that the individual would never become afflicted with osteoporosis, osteoporotic fractures, and the other conditions related to osteoporosis.

It would thus be desirable to provide an agent delivery system that facilitates minimally invasive administration of PTH-based agents. It would further be desirable to provide an agent delivery system that provides a pharmacokinetic profile of the PTH-based agent similar to that observed following subcutaneous administration.

### SUMMARY OF THE INVENTION

The present invention provides a method for preventing or treating osteopenia. The method comprises the steps of: providing a transdermal delivery device having disposed thereon at least one hPTH-based formulation and applying the transdermal device to a skin site of the patient to deliver hPTH to the patient.

In accordance with one embodiment of the invention, the transdermal device and HPTH formulation are selected to meet the following test: a device having a formulation disposed thereon achieves a mean Cmax value when applied to the thigh of the patient that is about 15% to about 75% of a mean Cmax value achieved by the same device and same formulation when applied to the abdomen of the patient under otherwise similar conditions.

In one embodiment, the device and formulation are selected to achieve a mean Cmax value when applied to the thigh of the patient that is about 20% to about 60% of a mean Cmax value achieved by the same device and formulation when applied to the abdomen of the patient. In yet another embodiment, the device and formulation are selected to achieve a mean Cmax value when applied to the thigh of the patient that is about 25% to about 35% of a mean Cmax value achieved by the same device and same formulation when applied to the abdomen of the patient. While the combination of the device and hPTH formulation selected according to the invention must meet the test wherein the Cmax achieved by application of the device to the thigh of a patient is between about 15% and about 75% of the Cmax achieved by application of the device to the abdomen of the same patient, in order to achieve the desired therapeutic effect according to the invention, the actual site of application of the selected device and formulation can be anywhere on the patient's body. In particular, and without limitation, the invention covers methods wherein the device is applied to the abdomen, thigh, or arm of the patient.

The selection of a particular site will depend on several factors. Factors that may be taken into account in selecting a site for applying the device according to the invention include a desired Cmax. For some patients, a lower Cmax may be desired which would indicate that an application to the thigh may be preferred. For other patients, a higher Cmax may be desired, and therefore applying the device to the abdomen of the patient may be preferred. In yet other instances, it may be advantageous to apply the transdermal device according to the invention to a site on the patient's skin other than the abdomen or the thigh. For example, for many patients, a device and formulation selected according to the invention may be advantageously applied to a site on the arm of the patient, for example, a site on the upper arm of the patient.

In one embodiment, the invention provides a method for preventing or treating osteopenia, comprising the steps of: providing a microprojection member having a plurality of stratum corneum-piercing microprotrusions; the microprojection member having a coating disposed thereon, the coating including at least one hPTH-based formulation; applying the microprojection member to a skin site of the patient, whereby the plurality of stratum corneum-piercing microprotrusions pierce the stratum corneum and deliver hPTH to the patient; and removing the microprojection member from the skin site. The microprojection member and the hPTH formulation are selected to meet the above test wherein a device comprising the microprojection member having a hPTH formulation disposed thereon achieves a mean Cmax value when applied to the thigh of the patient that is about 15% to about 75% of a mean Cmax value achieved by the same device and same formulation when applied to the abdomen of the patient under otherwise similar conditions.

In one embodiment, the device and formulation according to the invention are selected to achieve a mean plasma hPTH Tmax of 5 minutes or less.

In yet another embodiment, the device and formulation according to the invention are selected to achieve a hPTH mean plasma Cmax value of at least 50 pg/mL.

In a further embodiment, the device and formulation according to the invention are selected to achieve a HPTH mean plasma Cmax value of at least 100 pg/mL.

In still another embodiment, the device and formulation according to the invention are selected such that after 3 hours from applying the transdermal device to the patient's skin, the method achieves a hPTH plasma concentration of no more than about 10 pg/mL.

In a further embodiment, the device and formulation according to the invention are selected such that after 2 hours from applying the transdermal device to the patient's skin, the method achieves a HPTH plasma concentration of no more than about 20 pg/mL.

In still another embodiment, the device and formulation according to the invention are selected such that after 1 hour from applying the transdermal device to the patient's skin, the method achieves a HPTH plasma concentration of no more than about 30 pg/mL.

In yet another embodiment, the device and formulation according to the invention are selected such that the ratio between the Tmax achieved by the method and the Tmax achieved by subcutaneous injection of the hPTH is from about 1:2 to about 1:10.

In a further embodiment of the invention the device is applied to the abdomen of the patient and the ratio between the Tmax achieved by the method and the Tmax achieved by subcutaneous injection of the hPTH is from about 1:4 to about 1:6.

In still another embodiment, the device and formulation according to the invention are selected such when the device is applied to the skin of the patient for a period of about 30 minutes, the residual hPTH remaining on the device after application is about 40% to about 75% of hPTH present on the device prior to application of the device to the skin of the patient.

In one embodiment, the invention provides a method for preventing or treating osteopenia, comprising the steps of: providing a transdermal delivery device having disposed thereon at least one hPTH-based formulation; applying said transdermal device to a skin site located on the abdomen of said patient to deliver HPTH to said patient; wherein said formulation achieves a mean tmax value of 30 minutes or less.

In another embodiment, the invention provides a method for preventing or treating osteopenia, comprising the steps of providing a transdermal delivery device having disposed thereon at least one hPTH-based formulation; applying said transdermal device to a skin site located on the thigh of said patient to deliver hPTH to said patient; wherein said formulation achieves a mean tmax value of 30 minutes or less.

In yet another embodiment, the invention provides a method for preventing or treating osteopenia, comprising the steps of: providing a microprojection member having a plurality of stratum corneum-piercing microprotrusions; said microprojection member having a coating disposed thereon, said coating including at least one hPTH-based formulation; applying said microprojection member to a skin site located on the abdomen of said patient, wherein said formulation achieves a mean tmax value of 30 minutes or less.

In a still further embodiment, the invention provides a method for preventing or treating osteopenia, comprising the steps of: providing a microprojection member having a plurality of stratum corneum-piercing microprotrusions; said microprojection member having a coating disposed thereon, said coating including at least one hPTH-based formulation; applying said microprojection member to a skin site located on the thigh of said patient, wherein said formulation achieves a mean tmax value of 30 minutes or less.

In a further embodiment, the invention provides a method for preventing or treating osteopenia, comprising the steps of:providing a transdermal delivery device having disposed thereon at least one hPTH-based formulation comprising teriparatide (hPTH (1-34)) in a dose of approximately 40 μg; applying said transdermal device to a skin site of said patient to deliver hPTH to said patient; wherein said formulation achieves a mean tmax value of 30 minutes or less.

In another embodiment, the invention provides a method for preventing or treating osteopenia, comprising the steps of: providing a transdermal delivery device having disposed thereon at least one hPTH-based formulation comprising teriparatide (hPTH (1-34)) in a dose of approximately 40 μg; applying said transdermal device to a skin site of said patient to deliver hPTH to said patient; wherein said formulation achieves a mean tmax value of 30 minutes or less.

In yet another embodiment, the invention provides a method for preventing or treating osteopenia, comprising the steps of: providing a microprojection member having a plurality of stratum corneum-piercing microprotrusions; said microprojection member having a coating disposed thereon, said coating including at least one hPTH-based formulation comprising teriparatide (HPTH (1-34)) in a dose of approximately 40 μg; applying said microprojection member to a skin site of said patient, wherein said formulation achieves a mean tmax value of 30 minutes or less.

In yet another embodiment, the invention provides a method for preventing or treating osteopenia, comprising the steps of: providing a microprojection member having a plurality of stratum corneum-piercing microprotrusions; said microprojection member having a coating disposed thereon, said coating including at least one hPTH-based formulation comprising teriparatide (hPTH (1-34)) in a dose of approximately 40 μg; applying said microprojection member to a skin site of said patient, wherein said formulation achieves a mean tmax value of 30 minutes or less.

In other embodiments, the formulation achieves a mean tmax value of 20 minutes or less, a mean tmax value of 10 minutes or a mean tmax value of 5 minutes or less.

In one embodiment of the invention, the selected formulation comprises a hPTH-based agent selected from the group consisting of hPTH (1-34), hPTH salts and analogs, teriparatide and related peptides.

In a further embodiment of the invention, the hPTH salt is selected from group consisting of acetate, propionate, butyrate, pentanoate, hexanoate, heptanoate, levulinate, chloride, bromide, citrate, succinate, maleate, glycolate, gluconate, glucuronate,

3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

In a further embodiment of the invention, the formulation comprises teriparatide (hPTH (1-34)) in the range of approximately 10-100 μg.

In one embodiment of the invention the formulation comprises teriparatide (hPTH (1-34)) in a dose of approximately 10 μg.

In another embodiment of the invention, the formulation comprises teriparatide (hPTH (1-34)) in a dose of approximately 20 μg.

In still another embodiment of the invention, the formulation comprises teriparatide (hPTH (1-34)) in a dose of approximately 30 μg.

In still further embodiment of the invention, the formulation comprises teriparatide (hPTH (1-34)) in a dose of approximately 40 μg.

In one embodiment of the invention the method prevents or delays onset of osteoporosis.

In another embodiment, the method of prevents or delays the onset of osteoporotic fractures.

In yet another embodiment, the method reduces severity of osteoperosis deleterious effects.

In yet a further embodiment, the method reduces severity of osteoporotic fractures.

In one embodiment, the method prevents or delays the loss of bone mineral density.

In yet another embodiment, the method increases bone mineral density.

Accordingly, the present invention to provides a transdermal agent delivery apparatus and method that provides intracutaneous delivery of a PTH-based agent to a patient.

The present invention also provides a transdermal agent delivery apparatus and method that provides a pharmacokinetic profile of the PTH-based agent similar to or faster than that observed following subcutaneous administration.

The invention further provides a transdermal agent delivery apparatus and method that provides pharmacologically active blood concentration of a PTH-based agent for a period of up to eight hours.

The invention also provides a PTH-based agent formulation for intracutaneous delivery to a patient.

Also, the present invention provides a transdermal agent delivery apparatus and method that includes microprojections coated with a biocompatible coating that includes at least one biologically active agent, preferably, a PTH-based agent.

The present further provides a transdermal agent delivery apparatus that can be used to prevent or treat osteopenia in order to prevent or minimize the onset of osteoporosis, osteoporotic fractures, and other osteoporosis-related disorders.

Further, the invention provides methods, systems that allow delivery of hPTH with bioavailability that is similar to intravenous injection. Intravenous injection like bioavailability profiles obtained with the transdermal methods and systems of the invention are advantageous compared to other methods of delivery which do not achieve a pulsatile mode.

In accordance with the above objects and those that will be mentioned and will become apparent below, the apparatus and method for transdermally delivering a hPTH-based agent in accordance with one embodiment of the invention comprises a delivery system having a microprojection member (or system) that includes a plurality of microprojections (or array thereof) that are adapted to pierce through the stratum corneum into the underlying epidermis layer, or epidermis and dermis layers. The apparatus and method are for delivering a PTH-based agent to a patient to prevent or treat osteopenia. In a preferred embodiment, the microprojection member includes a biocompatible coating having at least one PTH-based agent disposed therein and administration to a patient prevents or treats osteopenia, and in one embodiment prevents or minimizes the onset and severity of osteoporosis, osteoporotic fractures, and other osteoporosis-related disorders.

In one embodiment of the invention, the microprojection member has a microprojection density of at least approximately 10 microprojections/cm², more preferably, in the range of at least approximately 200-2000 microprojections/cm².

In one embodiment, the microprojection member is constructed out of stainless steel, titanium, nickel titanium alloys, or similar biocompatible materials.

In another embodiment, the microprojection member is constructed out of a non-conductive material, such as a polymeric material. Alternatively, the microprojection member can be coated with a non-conductive material, such as Parylene®, or a hydrophobic material, such as Teflon®, silicon or other low energy material.

The coating formulations applied to the microprojection member to form solid biocompatible coatings can comprise aqueous and non-aqueous formulations. Preferably, the coating formulations include at least one PTH-based agent, which can be dissolved within a biocompatible carrier or suspended within the carrier.

In a preferred embodiment, the PTH-based agent is selected from the group consisting of hPTH(1-34), hPTH salts and analogs, teriparatide and related peptides. Throughout this application, the terms “PTH-based agent” and “hPTH(1-34) agent” include, without limitation, recombinant hPTH(I-34), synthetic hPTH(1-34), PTH(1-34), teriparatide, hPTH(1-34) salts, simple derivatives of hPTH(1-34), such as hPTH(1-34) amide, and closely related molecules, such as hPTH(1-33) or hPTH(1-31) amide, or any other closely related osteogenic peptide. Synthetic hPTH(1-34) is the most preferred PTH agent.

Examples of pharmaceutically acceptable hPTH salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

Preferably, the PTH-based agent is present in the coating formulation at a concentration in the range of approximately 1-30 wt. %. In some embodiments the PTH-based agent is in the range of 5-25 wt. %, or about 10-20 wt. %, or about 12.5-17.5 wt. %. In some embodiments the invention provides a concentration that contains at least about 1, 5, 7.5, 10, 12.5, 15, 17.5, 20, 22.5, 25, 27.5, or 29.9 wt. % PTH-based agent. In some embodiments the invention provides a concentration that contains no more than about 2, 5, 7.5, 10, 12.5, 15, 17.5, 20, 22.5, 25, 27.5, or 30 wt. % PTH-based agent.

Preferably, the amount of PTH-based agent contained in the solid biocompatible coating (i.e., microprojection member or product) is in the range of approximately 1 μg-1000 μg. In some embodiments the invention provides a composition that is in the range of 10-200 μg of PTH-based agent, or 10-100 μg of PTH-based agent, or about 10-90 μg of PTH-based agent, or about 10-80 μg of PTH-based agent, or about 10-70 μg of PTH-based agent, or about 10 -60 μg of PTH-based agent, or about 10-50 μg of PTH-based agent, or about 10-40 μg of PTH-based agent, or about 20-40 μg of PTH-based agent. In some embodiments the invention provides a composition that contains at least about 1, 5, 10, 15, 20, 25, 30, 35, 40, 45, 50, 55, 60, 65, 70, 75, 80, 85, 90, 95, 100, 110, 120, 130, 140, 150, 175, 200, 225, 250, 275, 300, 350, 400, 500, 600, 700, 800, 999.9 μg of PTH-based agent. In some embodiments the invention provides a composition that contains no more than 2, 5, 7.5, 10 15, 20, 25, 30, 35, 40, 45, 50, 55, 60, 65, 70, 75, 80, 85, 90, 95, 100, 110, 120, 130, 140, 150, 175, 200, 225, 250, 275, 300, 350, 400, 500, 600, 700, 800, 1000 μg of PTH-based agent.

Also preferably, the pH of the coating formulation is below approximately pH 6. More preferably, the coating formulation has a pH in the range of approximately pH 2-pH 6. Even more preferably, the coating formulation has a pH in the range of approximately pH 3-pH 6.

In certain embodiments of the invention, the viscosity of the coating formulation that is employed to coat the microprojections is enhanced by adding low volatility counterions. In one embodiment, the PTH-based agent has a positive charge at the formulation pH and the viscosity-enhancing counterion comprises an acid having at least two acidic pKas. Suitable acids include maleic acid, malic acid, malonic acid, tartaric acid, adipic acid, citraconic acid, fumaric acid, glutaric acid, itaconic acid, meglutol, mesaconic acid, succinic acid, citramalic acid, taritronic acid, citric acid, tricarballylic acid, ethylenediaminetetraacetic acid, aspartic acid, glutamic acid, carbonic acid, sulfuric acid and phosphoric acid.

Another preferred embodiment is directed to a viscosity-enhancing mixture of counterions, wherein the PTH-based agent has a positive charge at the formulation pH and at least one of the counterion comprises an acid having at least two acidic pKas. The other counterion comprises an acid with one or more pKas. Examples of suitable acids include hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

In the noted embodiments of the invention, the amount of counterion is preferably sufficient to neutralize the charge of the PTH. In such embodiments, the amount of the counterion or mixture of counterions is preferably sufficient to neutralize the charge present on the agent at the pH of the formulation. In additional embodiments, excess counterion (as the free acid or as a salt) is added to the peptide to control pH and provide adequate buffering capacity.

In another preferred embodiment, the agent comprises hPTH (1-34) and the counterion comprises a viscosity-enhancing mixture of counterions chosen from the group consisting of citric acid, tartaric acid, malic acid, hydrochloric acid, glycolic acid and acetic acid. Preferably, the counterions are added to the formulation to achieve a viscosity in the range of approximately 20-200 cp.

In a preferred embodiment of the invention, the viscosity-enhancing counterion comprises an acidic counterion, such as a low volatility weak acid that exhibits at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid.

In another preferred embodiment, the counterion comprises a strong acid that exhibits at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions, wherein at least one of the counterion comprises a strong acid and at least one of the counterion comprises a low volatility weak acid.

Another preferred embodiment is directed to a mixture of counterions, wherein at least one of the counterion comprises a strong acid and at least one of the counterion comprises a weak acid having a high volatility and exhibiting at least one pKa higher than about 2 and a melting point lower than about 50° C. or a boiling point lower than about 170° C. at Pₐₜₘ. Examples of such acids include acetic acid, propionic acid, pentanoic acid and the like.

The acidic counterion is preferably present in an amount that is sufficient to neutralize the positive charge present on the PTH-based agent at the pH of the formulation. In an additional embodiment, an excess counterion (as the free acid or as a salt) is added to control pH and to provide adequate buffering capacity.

In another embodiment of the invention, the coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

In one embodiment of the invention, the coating formulation includes at least one antioxidant, which can comprise sequestering agents, such sodium citrate, citric acid, EDTA (ethylene-dinitrilo-tetraacetic acid) or free radical scavengers, such as ascorbic acid, methionine, sodium ascorbate and the like. Presently preferred antioxidants comprise EDTA and methionine.

In the noted embodiments of the invention, the concentration of the antioxidant is preferably in the range of approximately 0.01-20 wt. % of the coating formulation. More preferably, the concentration of the antioxidant is in the range of approximately 0.02-10 wt. % of the coating formulation. Even more preferably, the concentration of antioxidant is in the range of approximately of 0.03-5 wt. % of the coating formulation.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives, such as sorbitan lauratealkoxylated alcohols, such as laureth-4 and polyoxyethylene castor oil derivatives, such as Cremophor EL®.

In the noted embodiments of the invention, the concentration of the surfactant is preferably in the range of approximately 0.01-20 wt. % of the coating formulation. Preferably, the concentration of the surfactant is in the range of approximately 0.05-5 wt. % of the coating formulation. More preferably, the concentration of surfactant is in the range of approximately of 0.1-2 wt. % of the coating formulation. In some embodiments of the invention the concentration of surfactant contains at least about 0.01, 0.02, 0.04, 0.06, 0.08, 0.10, 0.1.2, 0.14, 0.16, 0.18, 0.20, 0.22, 0.24, 0.26, 0.28, 0.3, 0.4, 0.5, 0.6, 0.7, 0.8, 0.9, 1.0, 2.0, 3.0, 4.0, 5.0, 10, 15, or 19.9 wt. % of the coating formulation. In some embodiments of the invention the concentration of surfactant contains at no more than about 0.02, 0.02, 0.04, 0.06, 0.08, 0.10, 0.1.2, 0.14, 0.16, 0.18, 0.20, 0.22, 0.24, 0.26, 0.28, 0.3, 0.4, 0.5, 0.6, 0.7, 0.8, 0.9, 1.0, 2.0, 3.0, 4.0, 5.0, 10, 15, or 20 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hydroxyethyl starch, carboxymethyl cellulose and salts of, dextran, poly(vinyl alcohol), poly(ethylene oxide), poly(2-hydroxyethyl-methacrylate), poly(n-vinyl pyrolidone), polyethylene glycol and mixtures thereof, and like polymers.

In a preferred embodiment, the concentration of the hydrophilic polymer in the coating formulation is in the range of approximately 1-30 wt. %, more preferably, in the range of approximately 1-20 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation is in the range of approximately 2-70 wt. %, more preferably, in the range of approximately 5-50 wt. % of the coating formulation, even more preferably, in the range of 10-30 wt. %. Most preferably, the concentration of the biocompatible carrier in the coating formulation is in the range of approximately 15-25 wt. %.hn some embodiments the invention provides a concentration that contains at least about 2, 5, 7.5, 10, 12.5, 15, 17.5, 20, 22.5, 25, 30, 35, 40, 50, or 69.9 wt. % biocompatible carrier. In some embodiments the invention provides a concentration that contains no more than about 3, 5, 7.5, 10, 12.5, 15, 17.5, 20, 22.5, 25, 30, 35, 40, 50, or 70 wt. % biocompatible carrier.

In another embodiment, the coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and insulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose and the like.

Preferably, the concentration of the stabilizing agent in the coating formulation is at a ratio of approximately 0.1-2.0:1 with respect to the PTH-based agent, more preferably, approximately 0.25-1.75:1 with respect to the PTH-based agent and even more preferably 0.5-1.50 with respect to the PTH-based agent.

The preferred PTH-based agent formulation has a composition of 15.5 wt. % hPTH(1-34), 16.6 wt. % sucrose, 0.2% wt. % polysorbate 20, and 0.03% wt. % EDTA made up in sterile water for injection and then brought to a pH of 5 with either 1 N hydrochloric acid or 1 N sodium hydroxide as needed.

The preferred PTH-based agent formulation dries to a solid state coating with the composition of 48 wt. % hPTH(1-34), 51.3 wt. % sucrose, 0.6% wt. % polysorbate 20, and 0.1% wt. % EDTA.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21 -succinaate sodium salt, paramnethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin. Most preferred solubilising/complexing agents are beta-Cyclodextrin, hydroxypropyl beta-Cyclodextrin, 2-hydroxypropyl-beta-Cyclodextrin and sulfobutylether7 beta-Cyclodextrin.

The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt. % to 50 wt. % of the coating formulation.

Preferably, the coating formulations have a viscosity less than approximately 500 centipoise and greater than 3 centipoise.

In one embodiment of the invention, the thickness of the biocompatible coating is less than 25 microns, more preferably, less than 10 microns, as measured from the microprojection surface.

In accordance with one embodiment of the invention, the method for delivering a PTH-based agent to a subject comprises (i) providing a microprojection member having a plurality of stratum comeum-piercing microprojections, the microprojection member having a biocompatible coating disposed thereon that includes at least one PTH-based agent, (ii) applying the microprojection member to a skin site on the subject, whereby the microprojections pierce the stratum corneum and deliver the PTH-based agent to the subject.

Preferably, the coated microprojection member is applied to the skin site via an impact applicator.

In one preferred embodiment, the coated microprojection member is applied to the upper arm. In another preferred embodiment, the coated microprojection member is applied to the abdomen. In still another preferred embodiment, the coated microprojection member is applied to the thigh.

Also preferably, the coated microprojection member is preferably left on the skin site for a period lasting from 5 seconds to 24 hours. Following the desired wearing time, the microprojection member is removed. In some embodiments, wherein the PTH-based agent is in the range of approximately 1 μg-1000 μg of the biocompatible coating. In one preferred embodiment, the PTH-based agent is approximately 20 μg of the biocompatible coating. In another preferred embodiment, the PTH-based agent is approximately 30 μg of the biocompatible coating. In still another preferred embodiment, the PTH-based agent is approximately 40 μg of the biocompatible coating.

Further, the pharmacokinetic profile of the transdermally delivered PTH-based agent is preferably at least similar to the pharmacokinetic profile observed following subcutaneous delivery.

In one preferred embodiment, the PTH-based agent is selected from the group consisting of hPTH (1-34), hPTH salts and analogs, teriparatide and related peptides. Also preferably, the HPTH salt is selected from group consisting of acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, fl-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate

In the methods of the invention, transdermal delivery of a PTH-based agent preferably exhibits rapid on-set of biological action. Also preferably, transdermal delivery of a PTH-based agent exhibits sustained biological action for a period of up to 8 hours.

In one embodiment, the transdermally delivered PTH-based agent comprises teriparatide (hPTH (1-34)) and the biocompatible coating comprises a dose of the PTH-based agent in the range of approximately 10-100 μg dose, wherein delivery of the PTH-based agent results in a plasma Cₘₐₓ of at least 50 pg/mL after one application.

In preferred embodiment, the transderrmally delivered PTH-based agent comprises teriparatide (hPTH (1-34)) and the biocompatible coating comprises a dose of the PTH-based agent in the range of approximately 10-100 μg dose, wherein delivery of the PTH-based agent results in a plasma Cₘₐₓ of at least 100 pg/mL after one application.

In a more preferred embodiment, the transdermally delivered PTH-based agent comprises teriparatide (HPTH (1-34)) and the biocompatible coating comprises a dose of the PTH-based agent in the range of approximately 10-100 μg dose, wherein delivery of the PTH-based agent results in a plasma Cₘₐₓ of at least 150 pg/mL after one application.

In a preferred embodiment, the transdermally delivered PTH-based agent comprises teriparatide (hPTH (1-34)) and the biocompatible coating comprises a dose of the PTH-based agent in the range of approximately 20 -40 μg dose, results in a Tmax of less than 5 minutes.

The invention also comprises a method of improving the pharmacokinetics of a transdermally delivered PTH-based agent comprising providing a microprojection member having a plurality of stratum comeum-piercing microprojections, the microprojection member having a biocompatible coating disposed thereon that includes at least one PTH-based agent and applying the microprojection member to a skin site on the subject, whereby the microprojections pierce the stratum comeum and deliver the PTH-based agent to the subject so that delivery of the PTH-based agent has improved pharmacokinetics compared to the pharmacokinetics characteristic of subcutaneous delivery.

In the noted embodiments, the improved pharmacokinetics can comprise increased bioavailability of the PTH-based agent. The improved pharmacokinetics can also comprise increased in Cₘₐₓ. Further, the improved pharmacokinetics can comprise decreased Tₘₐₓ. The improved pharmacokinetics can further comprise an enhanced absorption rate of the PTH-based agent.

The apparatus and method of the invention can thus be employed safely and effectively in the treatment of osteoporosis and bone fractures.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a pulsatile concentration profile, according to the invention;

FIG. 2 is a perspective view of a portion of one example of a microprojection member, according to the invention;

FIG. 3 is a perspective view of the microprojection member shown in FIG. 2 having a coating deposited on the microprojections, according to the invention;

FIG. 4 is a side sectional view of a microprojection member having an adhesive backing, according to the invention;

FIG. 5 is a side sectional view of a retainer having a microprojection member disposed therein, according to the invention;

FIG. 6 is a perspective view of the retainer shown in FIG. 4;

FIG. 7 is an exploded perspective view of an applicator and retainer, according to the invention;

FIG. 8 is a graph illustrating the charge profile for a PTH-based agent, according to the invention;

FIG. 9 is a graph illustrating the mole ratios of a net-charged species of a PTH-based agent, according to the invention;

FIG. 10 is a graph illustrating the mole ratios of acetic acid and the neutral form of a PTH-based agent, according to the invention;

FIG. 11 is a graph comparing plasma concentration of a PTH-based agent following transdermal delivery according to the invention and subcutaneous delivery;

FIG. 12 is a graph illustrating the aggregation percentage of a PTH-based agent with and without sucrose as a stabilizer, according to the invention;

FIG. 13 is a graph illustrating the oxidation of a PTH-based agent with and without antioxidants over time, according to the invention;

FIG. 14 is a graph illustrating the plasma concentration of a PTH-based agent following transdermal delivery, according to the invention;

FIG. 15 is a graph illustrating urinary concentrations of cAMP that reflects the bioavailability of a PTH-based agent, according to the invention;

FIG. 16 is a graph comparing plasma concentration of a PTH-based agent following transdermal according to the invention and subcutaneous delivery;

FIG. 17 is a graph illustrating the plasma concentration of a PTH-based agent following transdermal delivery to the thigh, upper arm or abdomen according to the invention and subcutaneous delivery to the thigh;

FIG. 18 is a graph illustrating the plasma concentration of a PTH-based agent following transdermal delivery to the thigh or abdomen according to the invention and subcutaneous delivery to the abdomen;

FIG. 19 is a graph illustrating serum corrected calcium concentration following transdermal delivery of a PTH-based agent, according to the invention;

FIG. 20 is a graph illustrating the urinary cAMP concentration following transdermal delivery of a PTH-based agent, according to the invention; and

FIG. 21 is a graph illustrating the urinary phosphate concentration following transdermal delivery of a PTH-based agent, according to the invention

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microproj ection” includes two or more such microproj ections and the like.

### DEFINITIONS

The term “transdermal”, as used herein, means the delivery of an agent into and/or through the skin for local or systemic therapy.

The term “transdermal flux”, as used herein, means the rate of transdermal delivery.

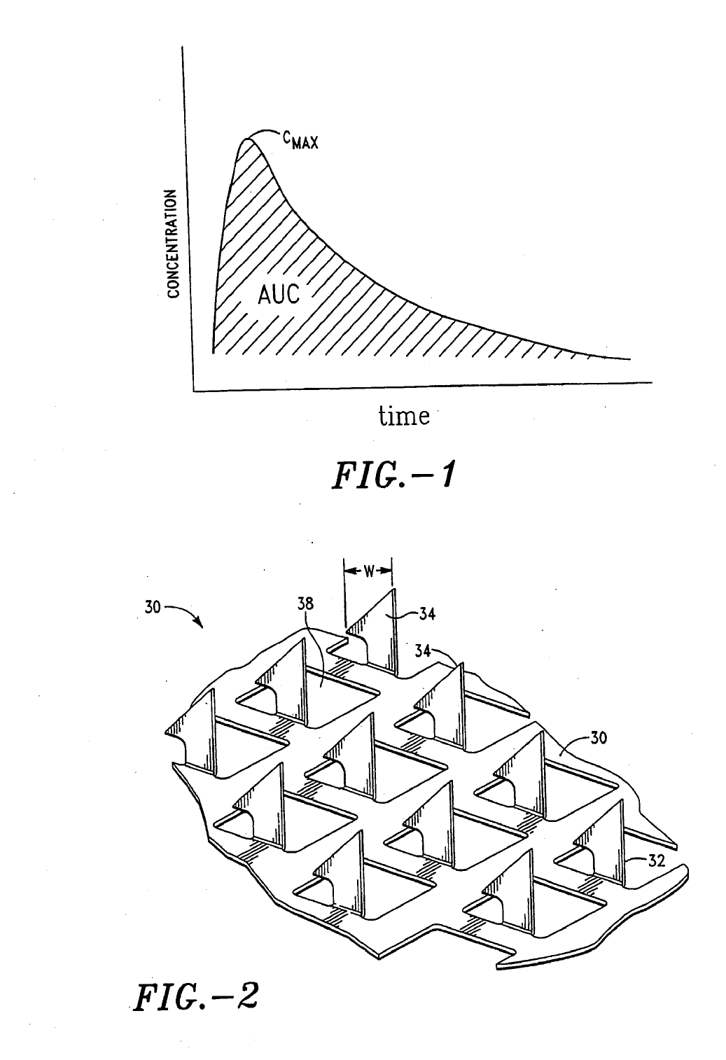

The terms “pulsatile delivery profile” and “pulsatile concentration profile”, as used herein, mean a post administration increase in blood serum concentration of a PTH-based agent from a baseline concentration to a concentration in the range of approximately 50-1000 pg/mL in a period ranging from 1 min. to 4 hr., wherein Cₘₐₓ is achieved, and a decrease in blood serum concentration from Cₘₐₓ to the baseline concentration in a period ranging from 1-8 hrs. after Cₘₐₓ has been achieved. As illustrated in FIG. 1, the noted concentration (or pharmacokinetic) profile typically reflects a rapid rise in blood serum concentration after administration (i.e., first region) and a slightly less rapid decline (i.e., second region) relative to the first region after Cₘₐₓ has been reached, which is generally reflected by a spike in the concentration profile.

Other concentration profiles resulting in a pulsatile delivery comprising a nse in blood concentration of the PTH-based agent to a Cₘₐₓ of 50-1000 pg/mL within a twelve-hour period following administration would also likely result in the desired beneficial effect and, hence, are within the scope of the present invention.

As discussed in detail herein, in one embodiment ofthe invention, the noted “pulsatile delivery profile” is reflected (or evidenced) by a curve of PTH-based agent concentration in the host's blood serum versus time having an area under the curve (AUC) in the range of approximately 14-5,240 pg h/mL and a Cₘₐₓ in the range of approximately 50-720 pg/mL for a microprojection member nominally containing 30 μg PTH(1-34).

The term “co-delivering”, as used herein, means that a supplemental agent(s) is administered transdermally either before the PTH-based agent is delivered, before and during transdermal flux of the PTH-based agent, during transdermal flux of the PTH-based agent, during and after transdermal flux of the PTH-based agent, and/or after transdermal flux of the PTH-based agent. Additionally, two or more PTH-based agents may be formulated in the coatings and/or formulations, resulting in co-delivery of the PTH-based agents.

The terms “PTH-based agent” and “hPTH(1-34) agent”, as used herein, include, without limitation, hPTH(1-34), hPTH salts, hPTH analogs, teriparatide, closely related peptides and agents having a peptide sequence that functions by the same means as the 34 N-terminal amino acids (the biologically active region) sequence of the 84-amino acid human parathyroid hormnone. The terms “PTH-based agent” and “hPTH(1-34) agent” thus include, without limitation, recombinant hPTH(1-34), synthetic hPTH(1-34), PTH(1-34), hPTH(1-34) salts, teriparatide, simple derivatives of hPTH(1-34), such as hPTH(1-34) amide and closely related molecules, such as hPTH(1-33) or hPTH(1-31) amide and closely related osteogenic peptides.

Examples of suitable hPTH salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

The noted PTH-based agents can also be in various forms, such as free bases, acids, charged or uncharged molecules, components of molecular complexes or nonirritating, pharmacologically acceptable salts.

It is to be understood that more than one PTH-based agent can be incorporated into the agent source, reservoirs, and/or coatings of this invention, and that the use of the term “PTH-based agent” in no way excludes the use of two or more such agents.

The terms “microprojections” and “microprotrusions”, as used herein, refer to piercing elements which are adapted to pierce or cut through the stratum comeum into the underlying epidermis layer, or epidermis and dermis layers, of the skin of a living animal, particularly a mammal and more particularly a human.

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

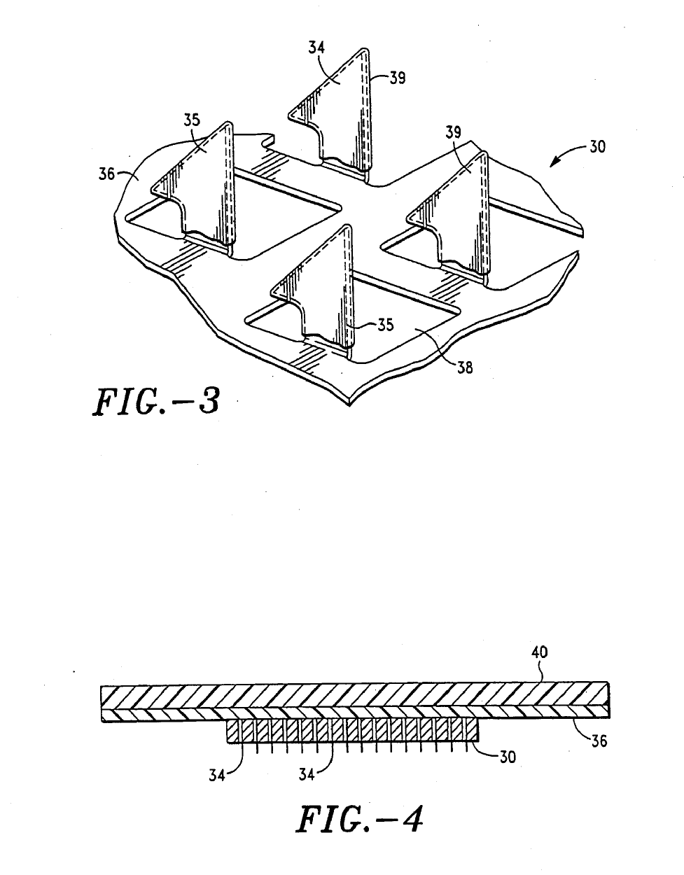

The term “microprojection member”, as used herein, generally connotes a microprojection array comprising a plurality of microprojections arranged in an array for piercing the stratum comeum. The microprojection member can be formed by etching or punching a plurality of microprojections from a thin sheet and folding or bending the microprojections out of the plane of the sheet to form a configuration, such as that shown in FIG. 2. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture that is employed to coat the microprojections and/or arrays thereof. Preferably, the coating formulation includes at least one PTH-based agent, which can be in solution or suspension in the formulation.

The term “biocompatible coating” and “solid coating”, as used herein, is meant to mean and include a “coating formulation” in a substantially solid state.

The present invention provides a method for preventing or treating osteopenia. The method comprises the steps of: providing a transdermal delivery device having disposed thereon at least one hPTH-based formulation and applying the transdermal device to a skin site of the patient to deliver HPTH to the patient.

In accordance with the invention, the transdermal device and hPTH formulation are selected to meet the following test: a device having a formulation disposed thereon achieves a mean Cmax value when applied to the thigh of the patient that is about 15% to about 75% of a mean Cmax value achieved by the same device and same formulation when applied to the abdomen of the patient under otherwise similar conditions.

In one embodiment, the device and formulation are selected to achieve a mean Cmax value when applied to the thigh of the patient that is about 20% to about 60% of a mean Cmax value achieved by the same device and formulation when applied to the abdomen of the patient. In yet another embodiment, the device and formulation are selected to achieve a mean Cmax value when applied to the thigh of the patient that is about 25% to about 35% of a mean Cmax value achieved by the same device and same formulation when applied to the abdomen of the patient. While the combination of the device and hPTH formulation selected according to the invention must meet the test wherein the Cmax achieved by application of the device to the thigh of a patient is between about 15% and about 75% of the cmax achieved by application of the device to the abdomen of the same patient, the actual site of application of the selected device and formulation can be anywhere on the patient's body. In particular, and without limitation, the invention covers methods wherein the device is applied to the abdomen, thigh, or arm of the patient. The selection of a particular site will depend on several factors. Factors that may be taken into account in selecting a site for applying the device according to the invention include a desired Cmax. For some patients, a lower Cmax may be desired which would indicate that an application to the thigh may be preferred. For other patients, a higher Cmax may be desired, and therefore applying the device to the abdomen of the patient may be preferred. In yet other instances, it may be advantageous to apply the transdermal device according to the invention to a site on the patient's skin other than the abdomen or the thigh. For example, for many patients, a device and formulation selected according to the invention may be advantageously applied to a site on the arm of the patient, for example, a site on the upper arm of the patient.

In one embodiment, the invention provides a method for preventing or treating osteopenia, comprising the steps of: providing a microprojection member having a plurality of stratum corneum-piercing microprotrusions; the microprojection member having a coating disposed thereon, the coating including at least one hPTH-based formulation; applying the microprojection member to a skin site of the patient, whereby the plurality of stratum comeum-piercing microprotrusions pierce the stratum corneum and deliver hPTH to the patient; and removing the microprojection member from the skin site. The microprojection member and the hPTH formulation are selected to meet the above test wherein a device comprising the microprojection member having a hPTH formulation disposed thereon achieves a mean Cmax value when applied to the thigh of the patient that is about 15% to about 75% of a mean Cmax value achieved by the same device and same formulation when applied to the abdomen of the patient under otherwise similar conditions.

As indicated above, one embodiment of the present invention comprises a delivery system including microprojection member (or system) having a plurality of microprojections (or array thereof) that are adapted to pierce through the stratum comeum into the underlying epidermis layer, or epidermis and dermis layers.

As discussed in detail herein, a key advantage of the present invention is that the delivery system delivers the PTH-based agent to a mammalian host, particularly, a human patient, whereby the PTH-based agent in the patient's serum after administration exhibits a preferred pulsatile concentration profile. The delivery system is farther amenable to self-administration of a 20 μg bolus dose of a PTH-based agent at least once daily.

Referring now to FIG. 2, there is shown one embodiment of a microprojection member 30 for use with the present invention. As illustrated in FIG. 2, the microprojection member 30 includes a microprojection array 32 having a plurality of microprojections 34. The microprojections 34 preferably extend at substantially a 90° angle from the sheet, which in the noted embodiment includes openings 38.

According to the invention, the sheet 36 can be incorporated into a delivery patch, including a backing 40 for the sheet 36, and can additionally include adhesive 16 for adhering the patch to the skin (see FIG. 4). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

As indicated, the microprojections 34 preferably have aprojection length less than 1000 microns. In one embodiment, the microprojections 34 have a projection length of less than 500 microns, more preferably, less than 250 microns. The microprojections 34 also preferably have a width in the range of approximately 25-500 microns and thickness in the range of approximately 10-100 microns.

In further embodiments of the invention, the biocompatibility of the microprojection member 30 can be improved to minimize or eliminate bleeding and irritation following application to the skin of a subject. Specifically, the microprojections 34 can have a length less than 145 microns, more preferably, in the range of approximately 50-145 microns, and even more preferably, in the range of approximately 70-140 microns. Also, the microprojection member 30 comprises an array preferably having a microprojection density greater than 100 microprojections/cm², and more preferably, in the range of approximately 200-3000 microprojections/cm². Further details regarding microprojection members having improved biocompatibility are found in U.S. application Ser. No. 11/355,729, which is hereby incorporated by reference in its entirety.

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymeric material. Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. application Ser. No. 10/880,701, which is incorporated by reference herein in its entirety.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

In certain embodiments of the invention, the microprojections 34 are preferably configured to reduce variability in the applied coating 35. Suitable microprojections generally comprise a location having a maximum width transverse to the longitudinal axis that is located at a position in the range of approximately 25% to 75% of the length of the microprojection from the distal tip. Proximal to the location of maximum width, the width of the microprojection tapers to a minimum width. Further details regarding the noted microprojection configurations are found in U.S. application Ser. No. 11/341,832, which is incorporated by reference herein in its entirety.

Referring now to FIG. 3, there is shown a microprojection member 30 having microprojections 34 that include a biocompatible coating 35 that includes a PTH-based agent. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. Pat. No. 6,855,372, which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are filly incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

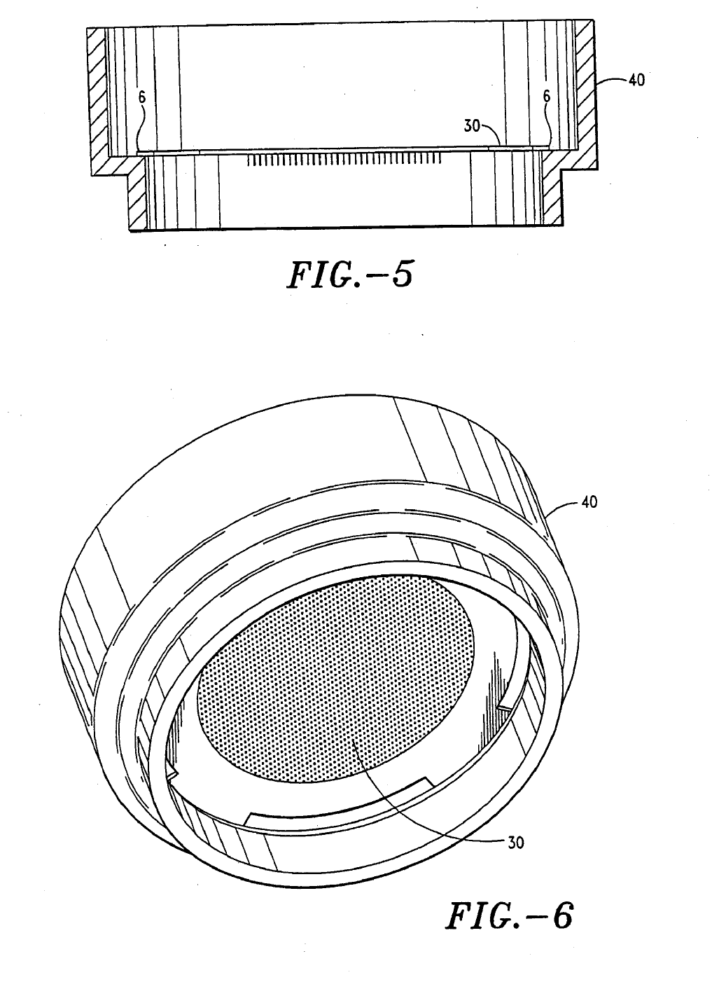

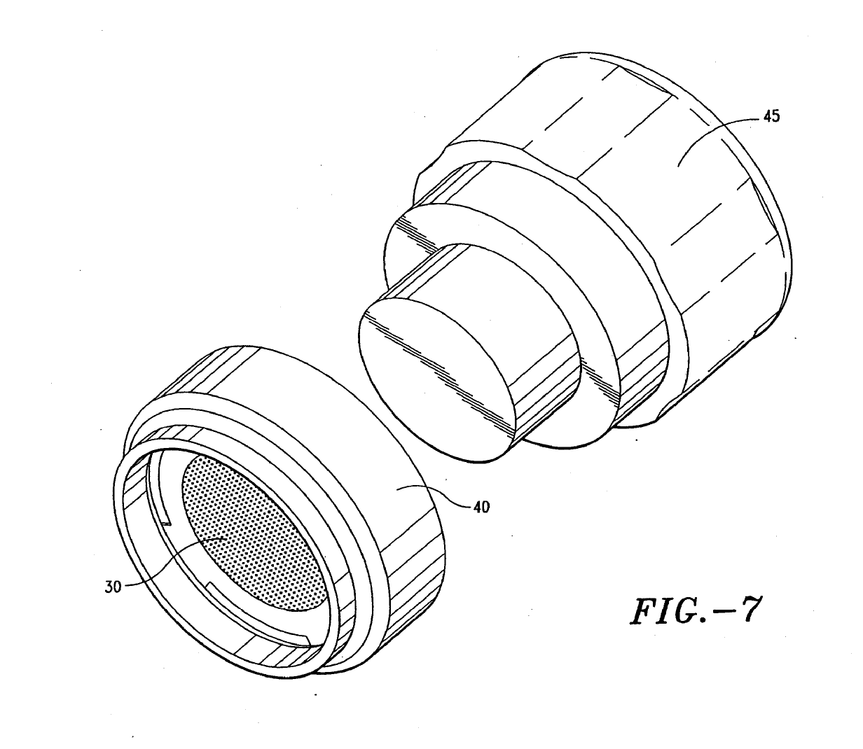

Referring now to FIGS. 5 and 6, for storage and application, the microprojection member 30 is preferably suspended in a retainer ring 40 by adhesive tabs 6, as described in detail in U.S. Pat. No. 6,855,131, which is incorporated by reference herein in its entirety.

After placement of the microprojection member 30 in the retainer ring 40, the microprojection member 30 is applied to the patient's skin. Preferably, the microprojection member 30 is applied to the patient's skin using an impact applicator 45, such as shown in FIG. 7 and described in U.S. Pat. No. 6,532,097, which is incorporated by reference herein in its entirety.

As indicated, according to one embodiment of the invention, the coating formulations applied to the microprojection member 30 to form solid biocompatible coatings can comprise aqueous and non-aqueous formulations having at least one PTH-based agent. According to the invention, the PTH-based agent can be dissolved within a biocompatible carrier or suspended within the carrier.

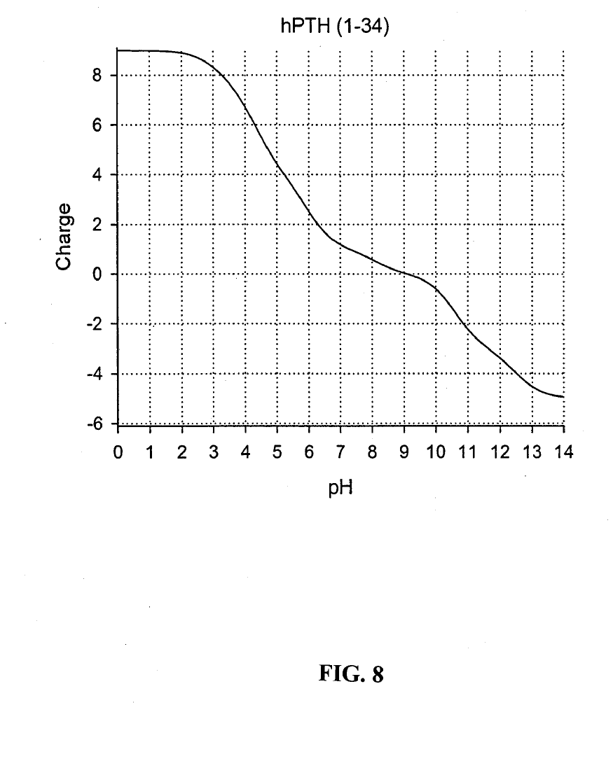

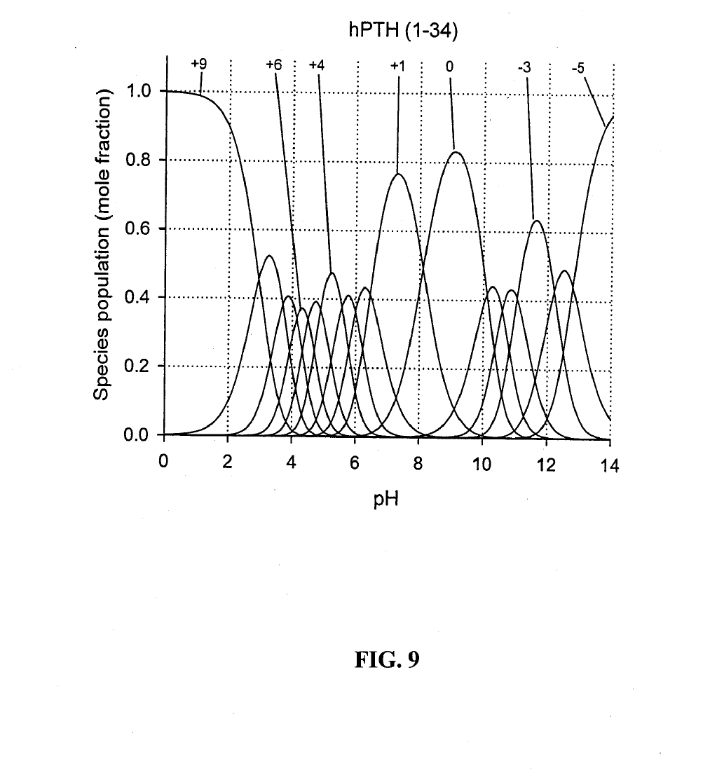

Referring now to FIG. 8, there is shown the predicted charge profile of hPTH(1-34), a peptide exhibiting 9 acidic pKa's and 6 basic pKa's. As illustrated in FIG. 8, the peptide presents a zero net electric charge at pH 9. This point is also called the isoelectric point or pI.

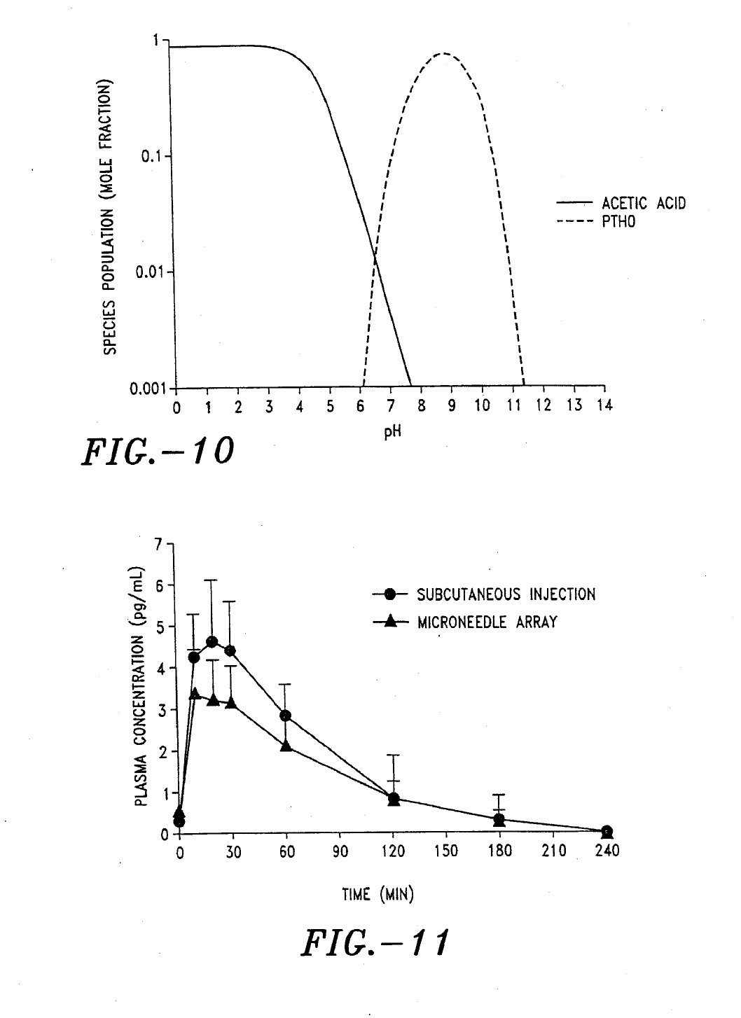

Referring now to FIG. 9, there is shown the predicted mole ratios of the net charged species of hPTH(1-34). As illustrated in FIG. 8, the neutral species only exist in significant amounts in the pH range of pH 6.5 to pH 11.5. In this pH range, the peptide has reduced water solubility and may precipitate out of solution. hPTH and closely related analogs thereof exhibit similar characteristics and behave similarly to hPTH (1-34).

The data thus reflects that hPTH(1-34) solubility that is compatible with formulations acceptable for coating on a microprojection array of the invention can be achieved at a pH below about pH 6 or above pH 11.5. Accordingly, in a preferred embodiment, the pH of the coating formulation is in the range of approximately pH 2-pH 6.

Referring now to FIG. 10, there is shown a superposition of the mole ratios for acetic acid and the neutral form of hPTH(1-34). The pH of a HPTH hexaacetate (mole ratio 1 to 6) in solution is about pH 5. At pH 5, negligible amounts of PTH are present as PTH zero net charge (PTH 0). The PTH is also highly soluble in water at concentrations in excess of 20%. During drying and subsequent storage, the free acetic acid will evaporate inherently resulting in formation of the water insoluble PTH 0. Subsequent reconstitution in water will not allow total solubilization of PTH. Accordingly, the use of a low volatility counterion provides a solid soluble formulation of PTH as long as the pH is maintained at least 2.5 pH units, preferably 3 pH units, below the pI of PTH. Preferably, this can be achieved by providing at least about two low volatility counterions to each molecule of PTH.

Therefore, in one embodiment of the invention, the coating formulations include a counterion or a mixture of counterions. Further, in the preferred pH range of pH 3-pH 6, the PTH-based agent will bear a positive charge.

In a preferred embodiment, the PTH-based agent is selected from the group consisting of hPTH(1-34), hPTH salts and analogs, teriparatide and related peptides, including, recombinant hPTH(1-34), synthetic hPTH(1-34), PTH(1-34), teriparatide, hPTH(1-34) salts, simple derivatives of hPTH(1-34), such as hPTH(1-34) amide, and closely related molecules, such as hPTH(l-33) or hPTH(1-31) amide, and any other closely related osteogenic peptide. Synthetic hPTH(1-34) is the most preferred PTH-based agent.

Examples of suitable hPTH salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

Preferably, the PTH-based agent is present in the coating formulation at a concentration in the range of approximately 1-30 wt. %. In some embodiments the PTH-based agent is in the range of 5-25 wt. %, or about 10-20 wt. %, or about 12.5-17.5 wt. % In some embodiments the invention provides a concentration that contains at least about 1, 5, 7.5, 10, 12.5, 15, 17.5, 20, 22.5, 25, 27.5, or 29.9 wt. % PTH-based agent. In some embodiments the invention provides a concentration that contains no more than about 2, 5, 7.5, 10, 12.5, 15, 17.5, 20, 22.5, 25, 27.5, or 30 wt. % PTH-based agent.

Preferably, the amount of PTH-based agent contained in the biocompatible coating on the microprojection member is in the range of 1-1000 μg. In some embodiments the invention provides a composition that is in the range of 10-200 μg of PTH-based agent, or 10-100 μg of PTH-based agent, or about 10 -90 μg of PTH-based agent, or about 10- 80 μg of PTH-based agent, or about 10-70 μg of PTH-based agent, or about 10 -60 μg of PTH-based agent, or about 10-50 μg of PTH-based agent, or about 10-40 μg of PTH-based agent, or about 20-40 μg of PTH-based agent. In some embodiments the invention provides a composition that contains at least about 1, 5, 10, 15, 20, 25, 30, 35, 40, 45, 50, 55, 60, 65, 70, 75, 80, 85, 90, 95, 100, 110, 120, 130, 140, 150, 175, 200, 225, 250, 275, 300, 350, 400, 500, 600, 700, 800, 999.9 μg of PTH-based agent. In some embodiments the invention provides a composition that contains no more than 2, 5, 7.5, 10 15, 20, 25, 30, 35, 40, 45, 50, 55, 60, 65, 70, 75, 80, 85, 90, 95, 100, 110, 120, 130, 140, 150, 175, 200, 225, 250, 275, 300, 350, 400, 500, 600, 700, 800, 1000 μg of PTH-based agent.

Preferably, the pH of the coating formulation is below about pH 6. More preferably, the coating formulation has a pH in the range of pH 2-pH 6. Even more preferably, the coating formulation has a pH in the range of approximately pH 3-pH 6.

In certain embodiments of the invention, the viscosity of the coating formulation is enhanced by adding low volatility counterions. In one embodiment, the PTH-based agent has a positive charge at the formulation pH and the viscosity-enhancing counterion comprises an acid having at least two acidic pKas. Suitable acids include, without limitation, maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid and phosphoric acid.

Another preferred embodiment is directed to a viscosity-enhancing mixture of counterions, wherein the PTH-based agent has a positive charge at the formulation pH and at least one of the counterions comprises an acid having at least two acidic pKas. The other counterion is an acid with one or more pKas. Examples of suitable acids include, without limitation, hydrochloric acid, hydrobromic acid, nitric acid, sulfuric acid, maleic acid, phosphoric acid, benzene sulfonic acid, methane sulfonic acid, citric acid, succinic acid, glycolic acid, gluconic acid, glucuronic acid, lactic acid, malic acid, pyruvic acid, tartaric acid, tartronic acid, fumaric acid, acetic acid, propionic acid, pentanoic acid, carbonic acid, malonic acid, adipic acid, citraconic acid, levulinic acid, glutaric acid, itaconic acid, meglutol, mesaconic acid, citramalic acid, citric acid, aspartic acid, glutamic acid, tricarballylic acid and ethylenediaminetetraacetic acid.

In the noted embodiments of the invention, the amount of counterion is preferably sufficient to neutralize the charge of the PTH. In such embodiments, the counterion or the mixture of counterion is preferably sufficient to neutralize the charge present on the agent at the pH of the formulation. In additional embodiments, excess counterion (as the free acid or as a salt) is added to the peptide to control pH and provide adequate buffering capacity.

In one preferred embodiment, the agent comprises hPTH (1-34) and the counterion comprises a viscosity-enhancing mixture of counterions chosen from the group consisting of citric acid, tartaric acid, malic acid, hydrochloric acid, glycolic acid and acetic acid. Preferably, the counterions are added to the formulation to achieve a viscosity in the range of about 20-200 cp.

In a preferred embodiment, the viscosity-enhancing counterion comprises an acidic counterion, such as a low volatility weak acid. Preferably, the low volatility weak acid counterion exhibits at least one acidic pKa and a melting point higher than about 50° C. or a boiling point higher than about 170° C. at Pₐₜₘ. Examples of such acids include, without limitation, citric acid, succinic acid, glycolic acid, gluconic acid, glucuronic acid, lactic acid, malic acid, pyruvic acid, tartaric acid, tartronic acid and fumaric acid.

In another embodiment, the counterion comprises a strong acid. Preferably, the strong acid exhibits at least one pKa lower than about 2. Examples of such acids include, without limitation, hydrochloric acid, hydrobromic acid, nitric acid, sulfonic acid, sulfuric acid, maleic acid, phosphoric acid, benzene sulfonic acid and methane sulfonic acid.

Another preferred embodiment is directed to a mixture of counterions, wherein at least one of the countenron comprises a strong acid and at least one of the counterions comprises a low volatility weak acid.

Another preferred embodiment is directed to a mixture of counterions, wherein at least one of the counterions comprises a strong acid and at least one of the counterions comprises a weak acid with high volatility. Preferably, the volatile weak acid counterion exhibits at least one pKa higher than about 2 and a melting point lower than about 50° C. or a boiling point lower than about 170° C. at Pₐₜₘ. Examples of such acids include, without limitation, acetic acid, propionic acid, pentanoic acid and the like.

The acidic counterion is preferably present in an amount sufficient to neutralize the positive charge present on the PTH-based agent at the pH of the formulation. In additional embodiments, excess counterion (as the free acid or as a salt) is added to control pH and to provide adequate buffering capacity.

In another embodiment of the invention, the coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

In one embodiment of the invention, the coating formulation includes at least one antioxidant, which can be sequestering agents, such sodium citrate, citric acid, EDTA (ethylene-dinitrilo-tetraacetic acid) or free radical scavengers such as ascorbic acid, methionine, sodium ascorbate and the like. Presently preferred antioxidants comprise EDTA and methionine.

In the noted embodiments of the invention, the concentration of the antioxidant is in the range of approximately 0.01-20 wt. % of the coating formulation. Preferably the antioxidant is in the range of approximately 0.02-10 wt. % of the coating formulation. Even more preferably, the concentration of antioxidant is in the range of approximately of 0.03-5 wt. % of the coating formulation.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, alkoxylated alcohols, such as laureth-4 and polyoxyethylene castor oil derivatives, such as Cremophor EL®.

In one embodiment of the invention, the concentration of the surfactant is in the range of approximately 0.01-20 wt. % of the coating formulation. Preferably the surfactant is in the range of approximately 0.05-5 wt. % of the coating formulation. More preferably, the concentration of surfactant is in the range of approximately of 0.1-2 wt. % of the coating formulation. In some embodiments of the invention the concentration of surfactant contains at least about 0.01, 0.02, 0.04, 0.06, 0.08, 0.10, 0.1.2, 0.14, 0.16, 0.18, 0.20, 0.22, 0.24, 0.26, 0.28, 0.3, 0.4, 0.5, 0.6, 0.7, 0.8, 0.9, 1.0, 2.0, 3.0, 4.0, 5.0, 10, 15, or 19.9 wt. % of the coating formulation. In some embodiments of the invention the concentration of surfactant contains at no more than about 0.02, 0.02, 0.04, 0.06, 0.08, 0.10, 0.1.2, 0.14, 0.16, 0.18, 0.20, 0.22, 0.24, 0.26, 0.28, 0.3, 0.4, 0.5, 0.6, 0.7, 0.8, 0.9, 1.0, 2.0, 3.0, 4.0, 5.0, 10, 15, or 20 wt. % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.01-20 wt. %, more preferably, in the range of approximately 0.03-10 wt. % of the coating formulation.

In another embodiment, the coating formulation includes a hydrophilic polymer selected from the following group: hydroxyethyl starch, carboxymethyl cellulose and salts of, dextran, poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 1-30 wt. %, more preferably, in the range of approximately 1-20 wt. % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose, stachyose, mannitol, and other sugar alcohols.

Preferably, the concentration of the biocompatible carrier in the coating formulation is in the range of approximately 2-70 wt. %, more preferably, in the range of approximately 5-50 wt. % of the coating formulation, even more preferably, in the range of 10-30 wt. %. Most preferably, the concentration of the biocompatible carrier in the coating formulation is in the range of approximately 15-25 wt. %. In some embodiments the invention provides a concentration that contains at least about 2, 5, 7.5, 10, 12.5, 15, 17.5, 20, 22.5, 25, 30, 35, 40, 50, or 69.9 wt. % biocompatible carrier. In some embodiments the invention provides a concentration that contains no more than about 3, 5, 7.5, 10, 12.5, 15, 17.5, 20, 22.5, 25, 30, 35, 40, 50, or 70 wt. % biocompatible carrier.

In another embodiment, the coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and inulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

Preferably, the concentration of the stabilizing agent in the coating formulation is at ratio of approximately 0.1-2.0:1 with respect to the PTH-based agent, more preferably, approximately 0.25-1.75:1 with respect to the PTH-based agent and even more preferably 0.5-1.50 with respect to the PTH-based agent.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, omipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the coating formulations and, hence, solid biocompatible coatings of the invention is particularly useful to prevent bleeding that can occur following application of the microprojection member or array and to prolong the pharmacokinetics of the PTH-based agent through reduction of the blood flow at the application site and reduction of the absorption rate from the skin site into the system circulation.

The concentration of the vasoconstrictor, if employed, is preferably in the range of approximately 0.1 wt. % to 10 wt. % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin. Most preferred solubilising/complexing agents are beta-Cyclodextrin, hydroxypropyl beta-Cyclodextrin, 2-hydroxypropyl-beta-Cyclodextrin and sulfobutylether7 beta-Cyclodextrin.

The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt. % to 50 wt. % of the coating formulation.

Other known formulation adjuvants can also be added to the coating formulations provided they do not adversely affect the necessary solubility and viscosity characteristics of the coating formulation and the physical integrity of the dried coating.

Preferably, the coating formulations have a viscosity less than approximately 500 centipoise and greater than 3 centipoise.

In one embodiment of the invention, the thickness of the biocompatible coating is less than 25 microns, more preferably, less than 10 microns, as measured from the microprojection surface.

The desired coating thickness is dependent upon several factors, including the required dosage and, hence, coating thickness necessary to deliver the dosage, the density of the microprojections per unit area of the sheet, the viscosity and concentration of the coating composition and the coating method chosen.

In accordance with one embodiment of the invention, the method for delivering a PTH-based agent contained in the biocompatible coating on the microprojection member includes the following steps: the coated microprojection member is initially applied to the patient's skin via an actuator, wherein the microprojections pierce the stratum comeum. The coated microprojection member is preferably left on the skin for a period lasting from 5 seconds to 24 hours. Following the desired wearing time, the microprojection member is removed.

Preferably, the amount of PTH-based agent contained in the biocompatible coating (i.e., dose) is in the range of approximately 1 μg-1000 μg, more preferably, in the range of approximately 10-200 μg per dosage unit. Even more preferably, the amount of PTH-based agent contained in the biocompatible coating is in the range of approximately 10-100 μg per dosage unit.

As stated, according to the invention, the PTH-based agent is delivered to the patient in a pulsatile fashion and, hence, exhibit pharmacokinetics resulting in a pulsatile concentration profile. In one embodiment of the invention, the pulsatile concentration profile is reflected (or evidenced) by a curve of PTH-based agent concentration in the host's blood serum versus time having an area under the curve (AUC) in the range of approximately 14-5,240 h·pg/mL and a Cₘₐₓ in the range of approximately 50-720 pg/mL for a microprojection member nominally containing 30 μg PTH(1-34).

In a further embodiment of the invention, the pulsatile concentration profile is reflected (or evidenced) by a curve of PTH-based agent concentration in the host's blood serum versus time having an area under the curve (AUC) in the range of approximately 140-5,240 h·pg/mL, Cₘₐₓ in the range of approximately 50 -720 pg/mL and Tₘₐₓ in the range of 5-30 min. for a microprojection member nominally containing 30 μg PTH(1-34).

In a presently preferred embodiment, a 20 μg bolus dose of a PTH-based agent is delivered in a pulsatile fashion by leaving the microprojection member in place for 30 minutes or less.

The noted pulsatile concentration profiles are preferably achieved via a PTH delivery regime in the range of 0.5 (i.e., once every other day)—2 pulses per day, more preferably, one full pulse (or dose) per day. However, as will be appreciated by one having ordinary skill in the art, the PTH can also be delivered via various additional dosing regimes.

In all cases, after a coating has been applied, the coating formulation is dried onto the microprojections 34 by various means. In a preferred embodiment of the invention, the coated microprojection member 30 is dried in ambient room conditions. However, various temperatures and humidity levels can be used to dry the coating formulation onto the microprojections. Additionally, the coated member can be heated, lyophilized, freeze dried or similar techniques used to remove the water from the coating.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by achieved in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods, such as sonophoresis or piezoelectric devices, can be used in conjunction with the invention.

### EXAMPLES

The following examples are given to enable those skilled in the art to more clearly understand and practice the present invention. They should not be considered as limiting the scope of the invention, but merely as being illustrated as representative thereof.

### Example 1

Delivery of hPTH (1-34) from coated microprojection arrays was evaluated in a hairless guinea pig (HGP) model. Microprojection arrays were produced using photo/chemical etching, and forming. The microprojection arrays used in this study were 2 cm² in area, with 320 microprojections/cm² and a projection length of 200 μm.

The microprojection arrays were coated with a 25% aqueous solution of hPTH (1 -34) at 40±10 μg per 2 cm² array, with a solid coating limited to the first 100 μm of the microprojections. Each coated microprojection array was assembled to a flexible polymeric adhesive backing. The resulting patch was assembled onto a retainer ring and loaded on a reusable impact applicator at the time of application to the HGP.

Each anesthetized HGP received a patch that was applied to a clean skin area for a wearing time of 1 hour. At various time intervals following patch application, blood samples were taken. Plasma hPTH (1-34) was determined by EIA, using a commercial enzyme immunoassay kit for HPTH from Peninsula Lab. (San Carlos, Calif.).

The plasma levels of HGPs receiving microprojection array patches coated with 40 μg of hPTH (1-34) were compared with subcutaneous (SC) administration of 20 μg of hPTH (1-34) (see FIG. 11).

An intravenous (IV) injection of 23 μg hPTH (1-34) was also performed in a separate group of 5 animals and the area under the curve (AUC) was used as a reference to calculate the total amounts absorbed/delivered following SC or microneedle array administration. The pharmacokinetic parameters of HPTH (1-34) following IV, SC, and microneedle array administration are shown in Table 1.

The pharmacokinetic (PK) profiles of immunoreactive hPTH(1-34) were similar for both SC and microprojection array delivery; tₘₐₓ (SC: 10 min vs 20 min, Cₘₐₓ (SC: 4.6±1.5 ng/mL vs 3.4±1.0 ng/ml); AUC_{240 min} (SC: 8.2±2.9 μg vs 6.6±1.8 μg) (n=10 per group, mean±SD).

The data indicate that hPTH(1-34) can be transdermally delivered with a PK profile similar to that of subcutaneous injection and highlight the feasibility of transdermal delivery of hPTH(1-34) using a microprojection array technology, which could be a more convenient alternative for osteoporotic patients.

TABLE 1

| Route of Administration | | |
|---|---|---|
| IV | SC | Array |

Single Dose Parameters

| | | | |
|---|---|---|---|
| Dose Amount (μg) | 22.5 | 19.5 | 40.0 |
| Dosage (μg/kg) | 30.9 | 29.2 | 52.8 |
| Fraction dose | 100 | 42 | 17 |
| absorbed/delivered (%) | | | |
| Cₘₐₓ (ng/mL) | 71.2 +/− 11.2 | 4.6 +/− 1.5 | 3.4 +/− 1.0 |
| Tₘₐₓ (min) | 1 | 20 | 10 |
| AUC (ng*h/ml) | 13.2 +/− 3.8 | 5.4 +/− 1.7 | 3.9 +/− 1.1 |
| Dose absorbed/ | | 8.2 +/− 2.9 | 6.6 +/− 1.8 |
| delivered (μg) | | | |

### Example 2

Example 2 demonstrates the utilization of a weak acid with a hPTH (1-34) agent to enhance the viscosity. The interaction of the weak acid anion with the positively charged a hPTH (1-34) agent leads to the formation of secondary bonds, e.g. hydrogen bonds, which results in an increase in solution viscosity. The greater the number of acidic groups, the greater the number of secondary bonds formed between the anions and the hPTH (1-34) agent, hence the greater the viscosity increase. Thus, the theoretical viscosity enhancing capabilities increase when monoacids, di-acids, tri-acids and tetra-acids are compared.

Various weak acid buffers have been incorporated in the hPTH (1-34) formulations in this experiment. A control formulation including PTH (1-34) actate with sucrose was also prepared. The experiment investigated the physicochemical properties afforded to hPTH (1-34) by various mixtures of mono-, di- and tri- acids and the stability of the solution formulations over a 48 hr period at 2-8° C. The PTH (1-34) formulations were buffered to a pH 5.2.

Referring now to Table 2, there is shown the viscosity results of the formulations. The citric and malic acid buffered formulations exhibited the largest increase viscosity enhancement compared to the control formulation (Lot No. 7528069A). Citric acid, a tri-acid, yielded a formulation with the highest viscosity.

The data reflected in Table 2 demonstrates that counterion mixtures of citric acid/acetic acid, malic acid/acetic acid, tartaric acid/acetic acid and hydrochloric acid/acetic acid increase the viscosity of HPTH (1-34) with respect to the control formulation of 20% PTH, 20% Sucrose, 0.2% Tween 20. Based on the results reflected in Table 2, the trend for viscosity enhancement following addition of weak acid buffers is preferably tri-acid to di-acid to mono-acid.

TABLE 2

| Formulation Lot No. | Viscosity (cP) |
|---|---|
| 20% PTH, 20% Sucrose, 0.2% Tween 20 | 68 |
| 20% PTH, 20% Sucrose, 0.5% HCl, | 87 |
| 0.2% Tween 20 | |
| 20% PTH, 20% Sucrose, 1.2% glycolic acid, | 53 |
| 0.2% Tween 20 | |
| 20% PTH, 20% Sucrose, 1.4% malic acid, | 116 |
| 0.2% Tween 20 | |
| 20% PTH, 20% Sucrose, 1.2% tartaric acid, | 77 |
| 0.2% Tween 20 | |
| 20% PTH, 20% Sucrose, 1.7% citric acid, | 172 |
| 0.2% Tween 20 | |

### Example 3

Example 3 demonstrates the utilization of a mixture of counterions with a hPTH(1-34) agent to enhance the dissolution of hPTH-based agent in vivo.

In a solid coating on a microprojection array, the agent is typically present in an amount of less than about 1 mg per unit dose. With the addition of excipients and counterions, the total mass of solid coating can be less than 3 mg per unit dose.

The array is usually present on an adhesive backing, which is attached to a disposable polymeric retainer ring. This assembly is typically packaged individually in a pouch or a polymeric housing. In addition to the assembly, this package contains an atmosphere (usually inert) that represents a volume of at least 3 mL. This large volume (as compared to that of the coating) acts as a sink for any volatile component. For example, at 20° C, the amount of acetic acid present in a 3 mL atmosphere as a result of its vapor pressure would be about 0.15 mg. This amount is typically what would be present in the solid coating if acetic acid were used as a counterion. In addition, components of the assembly, such as the adhesive, are likely to act as additional sinks for volatile components. As a result, during long-term storage, it is likely that the concentration of any volatile component present in the coating would change dramatically. These conditions are typical of packaging of pharmaceutical compounds where large amounts of excipients are usually present. Even with very potent biotechnology compounds that are lyophilized for use as an injectable, a very large excess of buffers and excipients is present in the dry cake.

In solution, or in the solid state, volatilization of the counterion occurs at the interface between the solution or the solid and the atmosphere. High diffusivity of solutes generally minimizes differences in concentration between the interface and the bulk of the solution. Conversely, in a solid state, diffusivity is very slow and greater concentration gradients of the volatile counterion are achieved between the interface and the bulk of the solution. Ultimately, the outer layer of the coating is depleted in counterion while the bulk of the solid coating is relatively unchanged, as compared to the initial dry state. This situation can result in a highly insoluble outer coating if the counterion is associated with an agent that is substantially insoluble in its neutral net charge state. Indeed, volatilization of the counterion results in formation of the water insoluble neutral species. This, in turn, jeopardizes dissolution of the agent from the solid coating upon exposure to the biological fluids. Accordingly, this experiment investigated the effect of adding low volatility counterions to improve coating solubility.

Several aqueous formulations containing hPTH (1-34) were prepared and are set forth in Table 3. These formulations contained the volatile counterion acetic acid. Certain formulations contained additional low volatility counterions hydrochloric acid, glycolic acid, or tartaric acid. The microprojection arrays (microprojection length 200 mm, 595 microprojections per array) had a skin contact area of approximately 2 cm². The tips of the microprojections were coated with the noted formulations by passing the arrays over a rotating drum carrying the PTH formulations, using the method and apparatus disclosed in U.S. Pat. No. 6,855,372, which is hereby incorporated by reference herein.

Four successive coatings were performed on each microprojection array at a temperature of 2-8° C. The amount of peptide coated on the arrays was evaluated by ultraviolet spectroscopy at a wavelength of 275 nm. Scanning electron microscopy revealed that the solid coating had a very smooth, glassy surface with no evidence of cracking. Furthermore, good uniformity of coating from microprojection to microproj ection was observed, with the coating limited to the first 100 μm of the microprojection tip.

Tip-coated arrays prepared in this manner were subsequently used for drug delivery studies in hairless guinea pigs (HGPs). HGPs were anesthetized by intramuscular injection of xylazine (8 mg/kg) and ketamine HCl (44 mg/kg). Anesthetized HGPs were catheterized through the carotid artery. The catheter was flushed with heparinized saline (20 IU/mL) to prevent clotting. The HGPs were maintained under anesthesia throughout the experiment via injection of sodium pentobarbital (32 mg/mL) directly into the catheter (0.1 mL/injection). Before application, blood samples were taken into heparinized vials (final concentration of heparin at 15 IU/mL), which served as 0 or baseline samples.

The application of the coated microprojection arrays was performed on the flank of the anesthetized animals with a spring-driven impact applicator (total energy=0.4 Joules, delivered in less than 10 milliseconds), of the type disclosed in U.S. Pat. No. 7,131,960, which is hereby incorporated in its entirety by reference herein. The system applied comprised a coated microprojection array device, adhered to the center of a LDPE backing with an adhesive (7 cm2 disc). Patches were retained on the skin for 1 h (n=4-5). A control group of animals (n=5) received an intravenous injection of 22 μg hPTH.

Blood samples were collected through the carotid catheter at time intervals following patch application. All blood samples were centrifuged immediately for plasma collection, the latter was then stored at −80° C. until analysis. Plasma hPTH(1-34) was determined by EIA, using a commercial enzyme immunoassay kit for HPTH from Peninsula Lab. (San Carlos, Calif.). The hPTH dose delivered by microprojection arrays was extrapolated based on the area under the curve (AUC) calculation compared to IV administration of hPTH.

As shown in Table 3, different amounts of PTH were delivered from each solid formulation. The solid formulations containing only PTH acetate delivered less than 2 mg on average. Addition of low volatility counterions to PTH acetate increased delivery significantly to up to 11.2 mg after the addition of the low volatility counterion glycolic acid. The two other non-counterions tested, i.e., tartaric and hydrochloric acid, also increased PTH delivery. Specifically, the counterion mixtures of glycolic acid/acetic acid, tartaric acid/acetic acid and hydrochloric acid/acetic acid increased the delivered amount of hPTH (1-34) with respect to the control formulation of 21.2% PTH, 3.8% acetic acid.

TABLE 3

| | Ratio | | |
|---|---|---|---|
| | (PTH:Acetate:low | Amount of PTH | Amount |
| Formulation | volatility | coated on array | delivered |
| solution (wt %) | counterion) | (μg) ± SD | (μg) ± SD |
| 21.2% PTH, | 1:3:0 | 28.0 ± 6.6 | 1.1 ± 1.1 |
| 3.8% acetic | | | |
| acid, water | | | |
| (q.s.) | | | |
| 21.2% PTH, | 1:3:0 | 35.0 ± 11.4 | 1.5 ± 1.7 |
| 3.8% acetic | | | |
| acid, water | | | |
| 22.3% PTH, | 1:2:2 | 40.0 ± 9.8 | 5.9 ± 2.5 |
| 2.7% acetic acid, | | | |
| 0.4% HCl, water | | | |
| 16.2% PTH, | 1:3:3 | 30.5 ± 2.3 | 6.1 ± 4.0 |
| 3.8% acetic acid, | | | |
| 0.5% HCl, | | | |
| 20.2% excipients, | | | |
| water. | | | |
| 6.2% PTH, | 1:3:4 | 45.9 ± 11.7 | 11.2 ± 2.7 |
| 3.8% acetic | | | |
| acid, 2.1% | | | |
| glycolic acid, | | | |
| 12.2% excipients, | | | |
| water. | | | |
| 16.2% PTH, | 1:3:2 | 29.0 ± 4.3 | 4.2 ± 1.5 |
| 3.8% acetic | | | |
| acid, 1.2% | | | |
| Tartaric acid, | | | |
| 20.23% | | | |
| excipients, | | | |
| water | | | |

### Example 4

Example 4 demonstrates the utilization of a stabilizing agent with a hPTH(1-34) agent to enhance the stability of the hPTH(1-34) agent.

Ten formulations, as shown in Table 4, were coated on titanium and monitored for chemical stability at 40° C. for a period of 60 days. The pH of the formulations containing the weak acid buffers was approximately pH 5.2, while the pH of the chloride containing formulations was approximately pH 5.4. The purity, oxidized PTH (1-34) product and soluble aggregates were monitored as a function of time by reverse phase high-pressure liquid chromatography (RPHPLC) and size exclusion chromatography (SEC), respectively. The results for each formulation are summarized in Tables 5-14.

The stability data generated suggests that the main mechanism of degradation of PTH in the solid state is via an aggregation process. Furthermore, the stability data indicates that addition of sucrose prevents aggregation of hPTH (1-34). FIG. 12 shows the percent aggregation of hPTH (1-34) formulations with and without sucrose at the 60-day time point.

TABLE 4

| Formulation | Formulation Composition (% w/w) |
|---|---|
| A | 20% PTH, 12.7% HCl |
| B | 20% PTH, 12.7% HCl, 0.01% EDTA |
| C | 20% PTH, 12.7% HCl, 1% methionine, 0.01% EDTA |
| D | 20% PTH, 12.7% HCl, 1.2% Tartaric acid, 1% |
| | methionine, 0.2% Tween 20, 0.01% EDTA |
| E | 20% PTH, 20% sucrose, 12.7% HCl, 0.2% Tween 20 |
| F | 20% PTH, 20% sucrose, 12.7% HCl, 0.2% Tween 20, |
| | 0.03% EDTA |
| G | 20% PTH, 20% sucrose, 12.7% HCl, 2% methionine, |
| | 0.2% Tween 20, 0.03% EDTA |
| H | 20% PTH, 20% sucrose, 1.2% Tartaric acid, 2% |
| | methionine, 0.2% Tween 20, 0.03% EDTA |
| I | 20% PTH, 20% sucrose, 1.2% Glycolic acid, 2% |
| | methionine, 0.2% Tween 20, 0.03% EDTA |
| J | 20% PTH, 20% sucrose, 1.7% Citric acid, 2% |
| | methionine, 0.2% Tween 20, 0.03% EDTA |

TABLE 5
Formulation Composition: 20% PTH, 12.7% HCL

| RP-HPLC | | | | | SEC | |
|---|---|---|---|---|---|---|
| Time | PTH Purity [%] | Total oxid [%] | Isomer [%] | Unknown[%] | Aggregation [%] | Unknown |
| (Days) | (% RSD) | (% RSD) | (% RSD) | (% RSD) | (% RSD) | [%] |
| 0 | 93.91(0.41) | 0.22 (7.87) | 4.40 (1.25) | 1.47 (22.55) | 0.10 (5.59) | 0.00 |
| 10 | 92.55 (0.82) | 0.27 (7.62) | 4.39 (0.88) | 2.79 (27.97) | 2.27 (66.38) | 0.39 |
| 24 | 89.76 (1.09) | 0.39 (12.76) | 4.45 (0.91) | 5.41 (18.04) | 4.73 (31.30) | 1.13 |
| 60 | 85.94 (0.47) | 0.38 (9.16) | 4.19 (1.18) | 9.49 (4.39) | 6.93 (5.34) | 3.22 |

TABLE 6
Formulation Composition: 20% PTH, 12.7% HCL, 0.01% EDTA

| RP-HPLC | | | | | SEC | |
|---|---|---|---|---|---|---|
| Time | PTH Purity [%] | Total oxid [%] | Isomer [%] | Unknown[%] | Aggregation [%] | Unknown |
| (Days) | (% RSD) | (% RSD) | (% RSD) | (% RSD) | (% RSD) | [%] |
| 0 | 93.75 (0.13) | 0.21 (7.39) | 4.33 (1.44) | 1.71 (5.00) | 0.15 (14.19) | 0.00 |
| 10 | 93.04 (0.19) | 0.25 (4.00) | 4.22 (0.96) | 2.48 (7.04) | 1.59 (15.78) | 0.12 |
| 24 | 91.51 (0.68) | 0.36 (13.89) | 4.41 (2.16) | 3.72 (15.63) | 2.66 (30.89) | 0.52 |
| 60 | 87.82 (0.70) | 0.37 (3.15) | 4.04 (3.73) | 7.77 (6.35) | 5.54 (3.62) | 1.97 |

TABLE 7
Formulation Composition: 20% PTH, 12.7% HCl, 0.01% EDTA, 1% Methionine

| RP-HPLC | | | | | SEC | |
|---|---|---|---|---|---|---|
| Time | PTH Purity [%] | Total oxid [%] | Isomer [%] | Unknown [%] | Aggregation [%] | Unknown |
| (Days) | (% RSD) | (% RSD) | (% RSD) | (% RSD) | (% RSD) | [%] |
| 0 | 93.77 (0.14) | 0.19 (2.99) | 4.29 (1.66) | 1.75 (3.73) | 0.14 (7.14) | 0.00 |
| 10 | 92.83 (0.59) | 0.51 (9.93) | 4.34 (1.80) | 2.32 (20.92) | 2.15 (44.83) | 0.32 |
| 24 | 90.69 (0.49) | 0.36 (18.73) | 4.46 (0.69) | 4.49 (9.64) | 3.01 (37.35) | 0.47 |
| 60 | 90.34 (0.71) | 0.36 (6.93) | 4.36 (10.62) | 4.94 (16.60) | 3.53 (20.6) | 0.79 |

TABLE 8
Formulation Composition: 20% PTH, 20% Sucrose, 12.7% HCl, 0.2% Tween20, 0.03% EDTA

| RP-HPLC | | | | | SEC | |
|---|---|---|---|---|---|---|
| Time | PTH Purity [%] | Total oxid [%] | Isomer [%] | Unknown [%] | Aggregation [%] | Unknown |
| (Days) | (% RSD) | (% RSD) | (% RSD) | (% RSD) | (% RSD) | [%] |
| 0 | 93.99 (0.38) | 0.45 (2.59) | 4.21 (1.35) | 1.26 (18.38) | 0.12 (12.39) | 0.00 |
| 10 | 92.98 (0.68) | 0.40 (28.67) | 4.25 (0.76) | 2.38 (30.89) | 0.40 (98.97) | 0.01 |
| 24 | 92.41 (0.06) | 0.54 (6.68) | 4.54 (0.34) | 2.51 (1.79) | 0.25 (10.58) | 0.00 |
| 60 | 91.88 (0.37) | 0.57 (1.75) | 4.24 (1.43) | 3.31 (8.41) | 0.88 (60.36) | 0.00 |

TABLE 9
Formulation Composition: 20% PTH, 1.2% Tartaric
acid, 0.01% EDTA, 1% Methionine, 0.2% Tween20

| RP-HPLC | | | | | SEC | |
|---|---|---|---|---|---|---|
| Time | PTH Purity [%] | Total oxid [%] | Isomer [%] | Unknown [%] | Aggregation [%] | Unknown |
| (Days) | (% RSD) | (% RSD) | (% RSD) | (% RSD) | (% RSD) | [%] |
| 0 | 93.79 (0.36) | 0.44 (11.53) | 4.30 (0.48) | 1.47 (18.95) | 0.13 (20.35) | 0.00 |
| 10 | 93.50 (0.08) | 0.34 (3.36) | 4.35 (1.09) | 1.81 (2.84) | 0.62 (131.08) | 0.01 |
| 24 | 91.40 (2.04) | 0.67 (7.90) | 4.34 (0.53) | 3.60 (53.01) | 2.10 (88.57) | 0.08 |
| 60 | 90.40 (0.03) | 0.66 (10.04) | 3.99 (2.75) | 4.95 (0.77) | 3.59 (28.55) | 0.33 |

TABLE 10
Formulation Composition: 20% PTH, 20% Sucrose, 12.7%
HCl, 0.2% Tween20, 0.03% EDTA, 2% Methionine

| RP-HPLC | | | | | SEC | |
|---|---|---|---|---|---|---|
| Time | PTH Purity [%] | Total oxid [%] | Isomer [%] | Unknown [%] | Aggregation [%] | Unknown |
| (Days) | (% RSD) | (% RSD) | (% RSD) | (% RSD) | (% RSD) | [%] |
| 0 | 93.92 (0.35) | 0.36 (3.24) | 4.10 (3.51) | 1.63 (10.64) | 0.15 (10.41) | 0.00 |
| 10 | 93.19 (0.67) | 0.36 (1.59) | 4.32 (1.67) | 2.13 (26.75) | 0.53 (106.67) | 0.03 |
| 24 | 92.66 (0.38) | 0.40 (15.94) | 4.55 (3.58) | 2.39 (8.32) | 0.26 (3.85) | 0.02 |
| 60 | 92.64 (0.17) | 0.39 (15.80) | 4.31 (3.04) | 2.66 (5.22) | 0.49 (48.12) | 0.02 |

TABLE 11
Formulation Composition: 20% PTH, 20% Sucrose, 1.2% Tartaric
acid, 0.2% Tween20, 0.03% EDTA, 2% methionine

| RP-HPLC | | | | | SEC | |
|---|---|---|---|---|---|---|
| Time | PTH Purity [%] | Total oxid [%] | Isomer [%] | Unknown [%] | Aggregation [%] | Unknown |
| (Days) | (% RSD) | (% RSD) | (% RSD) | (% RSD) | (% RSD) | [%] |
| 0 | 93.48 (0.12) | 0.35 (11.44) | 4.40 (0.47) | 1.77 (5.35) | 0.12 (9.90) | 0.01 |
| 10 | 93.50 (0.08) | 0.34 (3.36) | 4.35 (1.09) | 1.81 (2.84) | 0.62 (131.08) | 0.01 |
| 24 | 92.40 (0.44) | 0.37 (14.30) | 4.65 (2.28) | 2.58 (10.62) | 0.34 (37.00) | 0.01 |
| 60 | 91.83 (0.06) | 0.41 (5.12) | 4.49 (1.48) | 3.28 (2.13) | 0.36 (29.72) | 0.01 |

TABLE 12
Formulation Composition: 20% PTH, 20% Sucrose, 12.7% HCl, 0.2% Tween20

| RP-HPLC | | | | | SEC | |
|---|---|---|---|---|---|---|
| Time | PTH Purity [%] | Total oxid [%] | Isomer [%] | Unknown [%] | Aggregation [%] | Unknown |
| (Days) | (% RSD) | (% RSD) | (% RSD) | (% RSD) | (% RSD) | [%] |
| 0 | 93.76 (0.28) | 0.44 (2.27) | 4.20 (0.86) | 1.60 (13.80) | 0.14 (4.03) | 0.00 |
| 10 | 92.94 (0.29) | 0.29 (39.16) | 4.21 (1.58) | 2.56 (10.80) | 0.23 (26.45) | 0.00 |
| 24 | 92.58 (0.12) | 0.45 (3.14) | 4.61 (0.92) | 2.36 (5.99) | 0.51 (46.21) | 0.00 |
| 60 | 92.31 (0.05) | 0.47 (3.01) | 4.19 (1.69) | 3.03 (1.40) | 0.38 (11.16) | 0.00 |

TABLE 13
Formulation Composition: 20% PTH, 20% Sucrose, 1.2% Glycolic
acid, 0.2% Tween 20, 0.03% EDTA, 2% Methionine

| RP-HPLC | | | | | SEC | |
|---|---|---|---|---|---|---|
| Time | PTH Purity [%] | Total oxid [%] | Isomer [%] | Unknown [%] | Aggregation [%] | Unknown |
| (Days) | (% RSD) | (% RSD) | (% RSD) | (% RSD) | (% RSD) | [%] |
| 0 | 93.56 (0.12) | 0.40 (3.79) | 4.29 (1.08) | 1.74 (4.78) | 0.15 (13.33) | 0.00 |
| 10 | 93.41 (0.34) | 0.42 (8.43) | 4.28 (0.89) | 1.90 (16.54) | 0.37 (15.51) | 0.00 |
| 24 | 91.95 (0.90) | 0.51 (6.03) | 4.63 (1.14) | 2.92 (25.52) | 0.48 (42.42) | 0.00 |
| 60 | 91.85 (0.54) | 0.42 (2.73) | 4.47 (3.02) | 3.25 (16.05) | 0.82 (56.11) | 0.01 |

TABLE 14
Formulation Composition: 20% PTH, 20% Sucrose, 1.7%
Citric acid, 0.2% Tween20, 0.03% EDTA, 2% Methionine

| RP-HPLC | | | | | SEC | |
|---|---|---|---|---|---|---|
| Time | PTH Purity [%] | Total oxid [%] | Isomer [%] | Unknown [%] | Aggregation [%] | Unknown |
| (Days) | (% RSD) | (% RSD) | (% RSD) | (% RSD) | (% RSD) | [%] |
| 0 | 93.71 (0.34) | 0.37 (3.15) | 4.22 (1.54) | 1.70 (15.28) | 0.11 (10.19) | 0.00 |
| 10 | 93.63 (0.11) | 0.38 (14.65) | 4.23 (0.36) | 1.76 (5.71) | 0.22 (19.22) | 0.00 |
| 24 | 92.29 (0.21) | 0.35 (6.66) | 4.60 (1.95) | 2.76 (3.87) | 0.39 (23.47) | 0.00 |
| 60 | 90.29 (2.00) | 0.33 (9.09) | 4.48 (11.25) | 4.90 (34.30) | 2.14 (86.23) | 0.68 |

### Example 5

Example 5 demonstrates the utilization of an antioxidant to retard oxidation of hPTH(1-34) agent. Table 15 lists the seven formulations that were prepared for the stability study.

TABLE 15

| Formulation | Formulation Composition (% w/w) |
|---|---|
| A | 25% PTH |
| B | 25% PTH, 0.5% methionine |
| C | 25% PTH, 1% methionine |
| D | 25% PTH, 3% methionine |
| E | 25% PTH, 0.5 mM EDTA |
| F | 25% PTH, 1 mM EDTA |
| G | 25% PTH, 3 mM EDTA |

Table 16 highlights the results of a 3 month stability study. Three peaks detected by RPHPLC at Relative Retention Times of 0.36, 0.53 and 0.68 were attributed to oxidized species of hPTH(1-34) and are denoted Oxid 1, 2 and 3, respectively. In all cases, the Oxid 3 species was the predominant oxidation product.

TABLE 16
Oxidation (%)

| | Oxid 1 | Oxid 2 | Oxid 3 | Total |
|---|---|---|---|---|
| | RT = 0.36 | RT = 0.56 | RT = 0.68 | Oxid |
| Time Point 0 Months | | | | |
| Formulation | | | | |
| Control | 0.00 | 0.14 | 0.31 | 0.45 |
| 0.5% Methionine | 0.00 | 0.13 | 0.28 | 0.41 |
| 1% Methionine | 0.00 | 0.12 | 0.29 | 0.41 |
| 3% Methionine | 0.00 | 0.12 | 0.27 | 0.39 |
| 0.5 mM EDTA | 0.00 | 0.12 | 0.26 | 0.38 |
| 1 mM EDTA | 0.00 | 0.14 | 0.28 | 0.42 |
| 3 mM EDTA | 0.00 | 0.15 | 0.30 | 0.45 |
| Time Point 1 Months | | | | |
| Formulation | | | | |
| Control | 0.00 | 0.22 | 0.46 | 0.68 |
| 0.5% Methionine | 0.00 | 0.24 | 0.49 | 0.73 |
| 1% Methionine | 0.00 | 0.20 | 0.47 | 0.67 |
| 3% Methionine | 0.00 | 0.14 | 0.36 | 0.50 |
| 0.5 mM EDTA | 0.00 | 0.13 | 0.27 | 0.40 |
| 1 mM EDTA | 0.00 | 0.14 | 0.29 | 0.43 |
| 3 mM EDTA | 0.00 | 0.18 | 0.36 | 0.54 |
| Time Point 3 Months | | | | |
| Formulation | | | | |
| Control | 0.01 | 0.33 | 0.73 | 1.06 |
| 0.5% Methionine | 0.01 | 0.31 | 0.67 | 0.98 |
| 1% Methionine | 0.02 | 0.26 | 0.61 | 0.89 |
| 3% Methionine | 0.00 | 0.18 | 0.50 | 0.68 |
| 0.5 mM EDTA | 0.00 | 0.17 | 0.39 | 0.57 |
| 1 mM EDTA | 0.01 | 0.17 | 0.41 | 0.58 |
| 3 mM EDTA | 0.01 | 0.17 | 0.41 | 0.59 |

In summary, the formulation devoid of antioxidants yielded the highest percentage of total oxidized product and addition of methionine or EDTA retarded oxidation. The results indicate that methionine retards oxidation in a concentration dependent manner. However, EDTA did not exhibit this phenomenon. Addition of 0.5 mM EDTA to a formulation was as effective as 3 mM in retarding oxidation. Moreover, the results indicate that EDTA is more effectual in impeding oxidation than methionine.

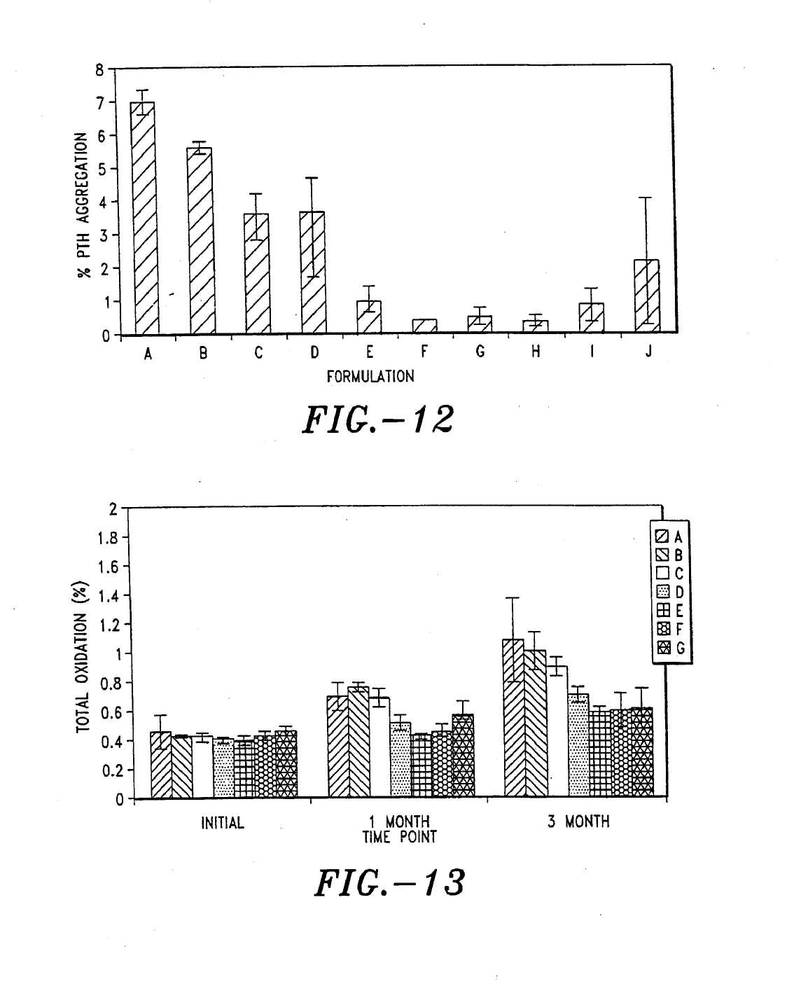

These results are graphically illustrated in FIG. 13, which provides the sum of oxidized species of hPTH (1-34).

### Example 6

A 2-part, phase 1, open-label study in healthy adult women was conducted to determine the pharmacokinetics and bioavialability of 30 μg hPTH(1-34) delivered by Macroflux TH0229 relative to subcutaneously administered (SC) FORTEO (teriparatide) 20 μg. Part 1 and Part 2 were each randomized, 2-treatment, 2-period, 2-way crossover studies with the treatments separated by at least five days. In Part 1, a dose finding study, each subject received a single 20 μg dose of SC FORTEO (teriparatide), injected in the thigh (Treatment A) and a single MACROFLUX® TH0229 system applied for 1 hour on the upper, outer arm (Treatment B). In Part 2, different subjects received a single 40 μg dose of SC FORTEO, injected in the thigh (Treatment C) and from 1 to 4 MACROFLUX TH0229 systems (depending on the amount of teriparatide absorbed in Part 1) applied for 1 hour (Treatment D). The number of MACROFLUX TH0229 systems used in Treatment D (Part 2) was determined by the amount of teriparatide absorbed in Part 1.

MACROFLUX TH0229 is a prototype microprojection array design with microprojections of 225 microns in length and a surface area of 2 cm² with 725 microprojections/cm². The microprojection arrays were applied to the outer, upper arm with 0.29 J/cm² impact force.

In the dose-finding phase, the majority of subjects had detectable plasma concentrations of teriparatide after MACROFLUX TH0229 dosing and undetectable plasma concentrations of teriparatide following SC FORTEO. 20 μg dosing. For this reason, the MACROFLUX TH0229 dosage was kept at a single application (nominally 30 μg) while the SC FORTEO. dosage was doubled to 40 μg in Part 2.

Plasma concentrations of teriparatide were measured in blood samples collected predose and 5, 10, 15, 30, and 45 minutes and 1, 1.25, 1.5, 2, 3, 4, 6, 8, 12, and 24 hours after dosing of Treatments A, B, C, and D.

Plasma concentrations of the biomarkers total calcium, ionized calcium and phosphate, as well as albumin and total protein, were measured in blood samples collected predose and 15, 30, and 45 minutes and 1, 1.25, 1.5, 2, 3, 4, 6, 8, 12, and 24 hours after dosing of Treatments A, C, and D. Biomarkers were not measured in Treatment B because of the uncertainty of drug delivery. Urine concentrations of creatinine, phosphate, and cAMP were measured in urine samples collected predose (within 2 hours before dosing) and collected and pooled by subject in the 0-2, 2-4,and 4-8 hour intervals after dosing of Treatments A, C, and D.

To compare the pharmacokinetics of Macroflux® hPTH (1-34) with that of the subcutaneous FORTEO®, dose-normalized AUC and Cₘₐₓ were calculated.
Cₘₐₓ=maximum observed plasma concentrations
Tₘₐₓ=time to maximum concentration
AUCₜ=area under the plasma concentration time profile from hour 0 to the last detectable concentration at time t was determined by the linear trapezoidal method
k=apparent elimination rate constant was estimated by linear regression of the log-transformed plasma concentrations during the terminal log-linear decline phase
t_{1/2}=apparent half-life (t_{1/2}) values was calculated as 0.693/k

AUCᵢₙ Macroflux® hPTH application to the thigh (40 μg) generally resulted in mean Cₘₐₓ and AUC values 36% and up to 25% lower, respectively, than that for application to the abdomen (30 and 40 μg)_{·f}=the AUC value extrapolated to infinity was calculated as the sum of AUCt, and the area extrapolated to infinity, calculated by the concentration at time t (Ct) divided by k. If, for any subject, k could not be estimated, the mean k for the treatment was used to estimate AUCinf for that subject.

The amount of teriparatide absorbed from the MACROFLUX TH0229 system was defined as follows:
(MACROFLUX AUCinf÷SC teriparatide AUCinf)*Dose of SC teriparatide

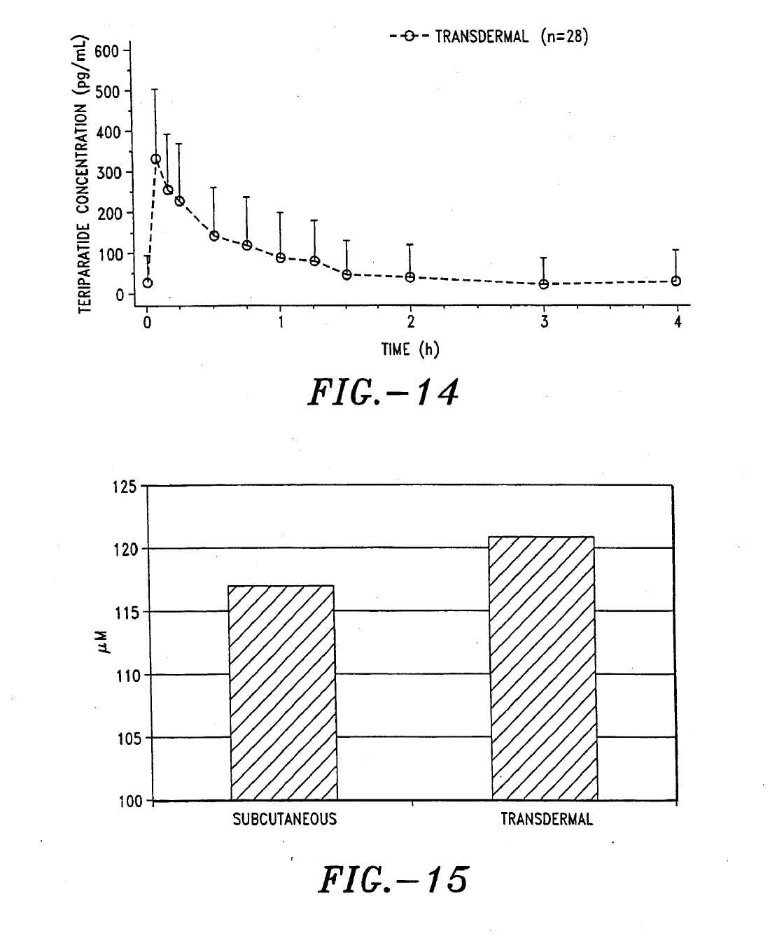

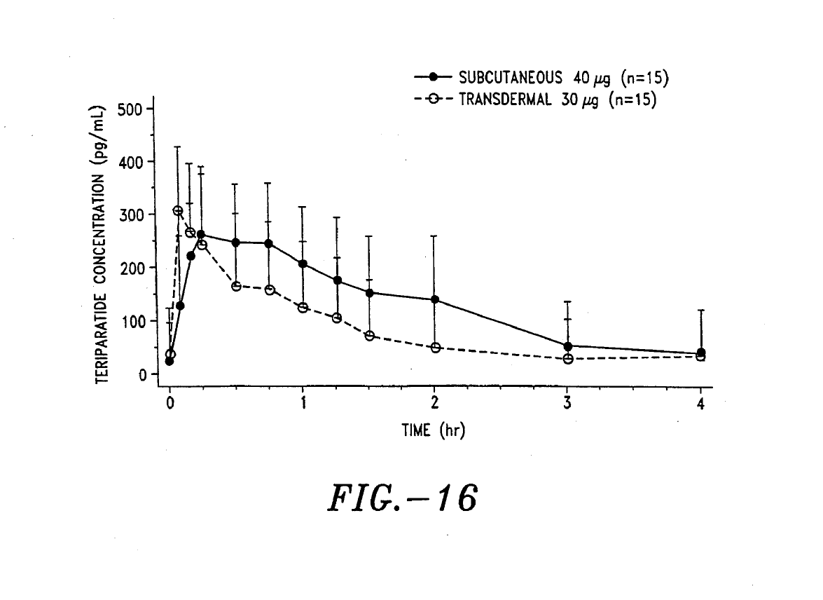

As shown in FIG. 14, transdermal delivery of a PTH-based agent yields effective absorption into the blood stream with the preferred pulsatile concentration profile of the PTH agent, i.e., rapid on-set and rapid off-set after reaching Cₘₐₓ. Further, as shown in FIG. 15, the biological activity of PTH following transdermal delivery is comparable to that following subcutaneous delivery as evidenced by increased levels of urinary cAMP excretion.

The plasma concentration of PTH following subcutaneous delivery and transdermal delivery is compared in FIG. 16, which further demonstrates rapid absorption following transdermal delivery. FIG. 16 similarly reflects a preferred pulsatile concentration profile of the PTH-based agent, i.e., rapid on-set and rapid off-set after reaching Cₘₐₓ.

The pharmacokinetic results of the subcutaneous and transdermal delivery are further provided in Table 17, which indicate similar bioavailability of PTH.

TABLE 17

| | | |
|---|---|---|
| | SC FORTEO | MACROFLUX TH0229 |
| Parameter | 40 μg (n = 20) | 30 μg (n = 20) |

| | | | | |
|---|---|---|---|---|
| Cₘₐₓ (pg/mL) | 167 | (120)^{a} | 305 | (120) |
| Tₘₐₓ (h) | 0.594 | (0.45)^{b} | 0.131 | (0.068) |
| t_{1/2} (h) | 1.40 | (0.66)^{c} | 0.99 | (0.77)^{d} |
| AUCₜ (pg · h/mL) | 494 | (910) | 661 | (1400) |
| AUC_{inf} (pg · h/mL) | 870 | (1100)^{b} | 837 | (1500) |

SC FORTEO, data dose-normalized to 30 μg
^{a}Five subjects' values were zero at each time point.
^{b}n = 15
^{c}n = 12
^{d}n = 16

Absorption of teriparatide was faster with MACROFLUX TH0229 than with SC FORTEO as demonstrated by the relatively higher dose-normalized mean Cₘₐₓ value (305 vs 167 pg/mL, respectively) and relatively smaller Tₘₐₓ value (0.13 vs 0.59 hours, respectively). The mean terminal half-life for teriparatide was also shorter with MACROFLUX TH0229 (0.99 hours) than with SC FORTEO. (1.4 hours).

In Part 1, SC FORTEO. treatment resulted in significant changes in some, but not all, biomarkers, for example serum phosphate was significantly decreased (p=0.0065) and adjusted urinary cAMP was significantly increased (p=0.0468) following dosing. In Part 2, with the doubled dosage of SC FORTEO. (40 μg), both treatments showed the expected patterns of biomarker activity relative to predose: significantly increased concentrations of serum total calcium, ionized calcium, and corrected calcium, and adjusted urinary cAMP and significantly reduced concentrations of serum phosphate. See Tables 18 and 19. Increase in adjusted urinary phosphate concentrations was significant for SC FORTEO. (p=0.0064) but not significant with MACROFLUX TH0229. No significant treatment differences were seen in change from predose concentrations of any of these analytes

TABLE 18

| | SC FORTEO. | MACROFLUX TH0229 |
|---|---|---|
| | 40 μg | (nominally 30 μg) |
| Biomarker | (n = 20) | (n = 20) |
| Total calcium (mmol/L) | 0.060^{a} | 0.044^{a} |
| Corrected calcium (mmol/L) | 0.042^{a} | 0.028^{a} |
| Ionized calcium (mmol/L) | 0.021^{a} | 0.019^{a} |
| Phosphate (mmol/L) | −0.101^{b} | −0.075^{b} |

^{a}Difference between predose and maximal value after dosing
^{b}Difference between predose and minimal value after dosing

TABLE 19

| | SC FORTEO. | TMACROFLUX H0229 |
|---|---|---|
| | 40 μg | (nominally 30 μg) |
| Biomarker | (n = 20) | (n = 20) |
| Adjusted urinary | 106^{a} | 107^{a} |
| cAMP (μmol/L) | | |
| Adjusted urinary | 32.0^{a} | 15.7^{a} |
| phosphate (mmol/L) | | |

^{a}Difference between predose (Hour −2 to 0) and postdose (Hour 0 to 2)

No serious adverse events (SAEs) were reported, and no subject discontinued from the study because of an adverse event (AE). AEs were reported by 50% (16/32) of those taking MACROFLUX TH0229, by 70% (14/20) of those taking SC FORTEO 40 μg, and by 33% (4/12) of those taking SC FORTEO. 20 μg. The AEs reported in this study were mild or moderate in severity, and the majority have been previously reported for teriparatide. The most common AEs were headache, nausea, and dizziness. No clinically significant changes were observed for vital signs, clinical laboratory test results, ECG results, or physical examination findings during the study.

### Example 7

A phase 1, open-label, randomized, crossover study of Macroflux hPTH patch, 30 μg and teriparatide PTH (Forteo™) was conducted in twenty four healthy postmenopausal women. The purpose of the study was to characterize the pharmacokinetic and pharmacodynamic properties of an application site for Macroflux hPTH patch, 30 μg. Additionally, tolerability and the topical and systemic safety of Macroflux hPTH were also evaluated. Three application locations were tested: thigh, upper arm, and abdomen. A 20 μg subcutaneous (SC) injection of Forteo™ was used as a control and was injected into the thigh opposite to the Macroflux hPTH application. The subjects, between the ages of 45 and 85, were treated once per day, on four consecutive days in a randomized fashion. The Macroflux microprojection arrays used in the clinical study have microprojection length of 200 μm and a surface area of 2 cm² with 725 microprojections/cm². The microprojection arrays were applied with a force of 0.20 J/cm² and left in place for thirty minutes.

To compare the pharmacokinetics of Macroflux® hPTH (1-34) among the 3 application sites with that of the SC FORTEO®, dose-normalized AUC and Cₘₐₓ were calculated. Plasma concentrations of hPTH were measured in blood samples collected at 0 (predose), 5, 10, 15, and 30 minutes, 1, 2, 3, 4, and 8 hours after dosing initiation.

Plasma hPTH (1-34) concentration as a function of time following Macroflux® hPTH was plotted and compared to that of SC FORTEO® hPTH (1-34). The following pharmacokinetic parameters including AUC_{inf}, Cₘₐₓ, Tₘₐₓ, and t_{1/2} were calculated for each treatment and by subject.

Serum concentrations ofthe biomarkers total calcium, ionized calcium, phosphate, albumin, and total protein were measured in blood samples collected at 0 (predose), 1, 2, 3, 4, and 8 hours after dosing initiation.

Serum concentrations of total and ionized calcium, corrected calcium, phosphate, albumin, and total protein were obtained at each measured time point for all treatment groups and descriptive statistics presented.

Urine concentrations of creatinine, phosphate, and cAMP were measured in urine samples collected and pooled by subject at four time intervals, pre-dose (within 2 hours before dosing) and in the 0.2, 2-4, and 4-8 hour intervals after dosing. Descriptive statistics was presented for urinary concentrations of cAMP and phosphate, each adjusted for creatinine concentration, at each measured time point for all treatments. Cyclic AMP and phosphate measurements were presented as a ratio to creatinine.

Mean changes from baseline were calculated for each parameter and compared between treatment groups.

Descriptive statistics were calculated for the pharmacokinetic and pharmacodynamic parameters described above and were compared among the treatment groups. For exploratory analysis of treatment difference, a mixed-effect analysis of variance (ANOVA) model was fitted. The model included treatment, treatment sequence and period as fixed effects and subject-within-sequence as a random effect. The least square estimates of the treatment ratio of mean pharmacokinetic parameters (logtransformed AUC and Cₘₐₓ) and the 90% confidence interval were computed.

Serum anti-hPTH (1-34) antibody levels were measured in blood samples collected predose on Day 1 and at the follow-up visits on Day 18 and Day 32 (study termination/study completion).

TABLE 20

| | Treatment | | | |
|---|---|---|---|---|
| | Mean ± SD, n = 24 | | | |
| | Macroflux ® hPTH (1-34) | | | SC FORTEO ® |
| | 30 μg | | | 20 μg |
| Parameter | Thigh | Upper Arm | Abdomen | Thigh |
| Cₘₐₓ (pg/mL) | 56.9 ± 27.8 | 96.4 ± 63.8 | 106.9 ± 39.6 | 54.3 ± 21.8 |
| Tₘₐₓ (h) | 0.14 ± 0.10 | 0.14 ± 0.09 | 0.15 ± 0.06 | 0.39 ± 0.24 |
| t_{1/2} (h) | 0.80 ± 0.49 | 0.53 ± 0.16 | 0.80 ± 0.35 | 1.39 ± 0.51 |
| AUCₜ (pg · h/mL) | 29.9 ± 24.0 | 46.5 ± 35.9 | 65.7 ± 38.0 | 81.7 ± 38.9 |
| Relative Bioavailability ^{a} (%) | 37.4 ± 23.8 | 56.3 ± 42.8 | 92.1 ± 60.4 | REFERENCE |
| AUC₍₀₋₈₎ (pg · h/mL) | 36.4 ± 24.7 | 54 ± 36.5 | 77.5 ± 38.9 | 103.8 ± 50.7 |
| Relative Bioavailability ^{a} (%) | 37.6 ± 23.0 | 56.4 ± 38.1 | 89.3 ± 55.1 | REFERENCE |
| AUC_{inf} (pg · h/mL) | 46.6 ± 26.4 | 60.2 ± 34.9 | 88.2 ± 40.6 | 117.4 ± 38.8 |
| Relative Bioavailability ^{a} (%) | 40.3 ± 20.1 | 51.7 ± 27.1 | 81.5 ± 46.3 | REFERENCE |

^{a} Presented using the calculation without dose normalization. AUC_{inf} could not be accurately estimated for some subjects, thus relative bioavailability is presented based on AUCₜ, AUC₍₀₋₈₎ and AUC_{inf}.

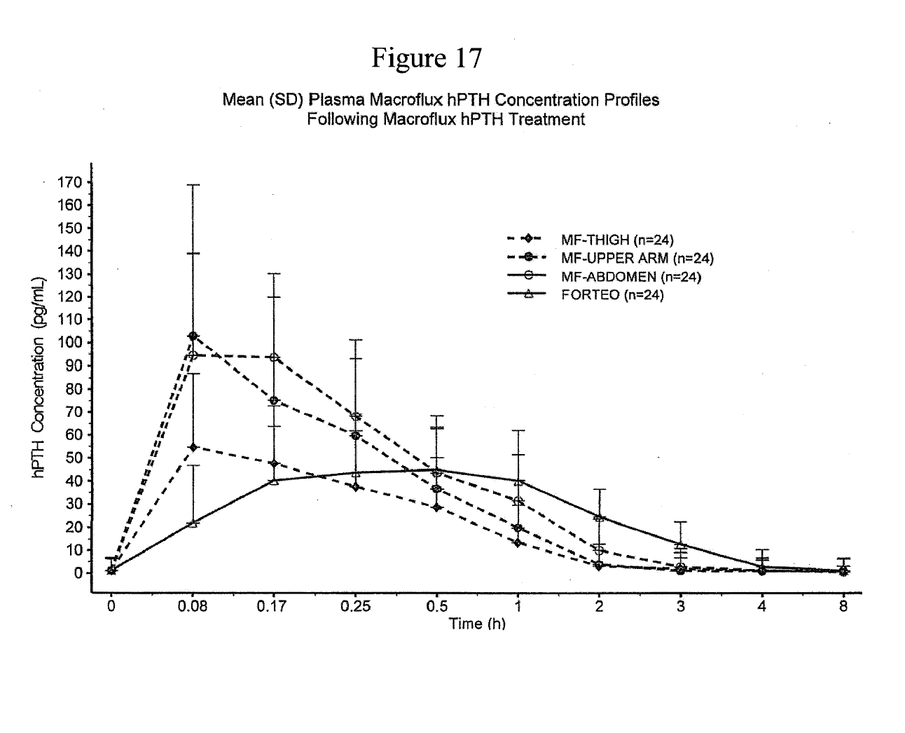

The three application sites (abdomen, thigh, and upper arm) for Macroflux® hPTH application had comparable Tₘₐₓ and terminal half life. See FIG. 17 and Table 20. Application of Macroflux® hPTH (30 μg) to the abdomen achieved comparable relative bioavailability (˜92%) to SC FORTEO® 20 μg injection in the thigh but with higher Cₘₐₓ (˜197%). Macroflux® HPTH (30 μg) application to the thigh achieved comparable Cₘₐₓ (˜105%) to SC FORTEO® 20 μg to the thigh but with lower relative bioavailability (37%). Macroflux® hPTH (30 μg) application to the arm achieved higher Cₘₐₓ (˜177%) but with lower relative bioavailability (56%) as compared to SC FORTEO® 20 μg. The mean terminal half-life for teriparatide was shorter with Macroflux® hPTH application (0.5 to 0.8 hours) than with SC FORTEO® (1.4 hours). With all Macroflux® hPTH treatments, Tₘₐₓ occurred earlier than with SC FORTEO® (8.5 min versus 23 min, respectively). See FIG. 17 and Table 20.

Macroflux®V hPTH treatment resulted in significant changes in some but not all biomarkers. Both SC FORTEO® and Macroflux® hPTH treatments showed the expected patterns for biomarker activity relative to predose. Serum corrected calcium significantly increased for all treatment groups with maximum concentration increases at 4 hours (p<0.05 for all time points and treatments compared to pretreatment). The mean maximum increases were 0.090±0.060 (thigh), 0.063±0.058 (upper arm), and 0.075±0.050 (abdomen) mmol/L with Macroflux hPTH and 0.105±0.153 mmol/L with SC FORTEO. Adjusted urinary cAMP increased for all treatment groups at 2 hours compared to pretreatment (p<0.003). Increases in post-dose concentrations (approximately 4 hours) of serum total calcium were significant with SC FORTEO® and with Macroflux® hPTH treatments to the thigh and abdomen but not at the upper arm. Significant increases in serum ionized calcium occurred with SC FORTEO® and after Macroflux® hPTH application to the thigh only. Adjusted urinary phosphate concentrations increased from predose values after both Macroflux@ HPTH applications (all sites) and SC FORTEO® injection (p<0.0001). None of the treatments resulted in the expected reduced concentrations of serum phosphate. No treatment differences were seen in the change from predose concentrations of serum albumin and total protein.

Twenty-four subjects were enrolled, and all subjects completed all study treatments. No serious adverse events (SAEs) were reported, and no subject discontinued from the study because of an adverse event (AE). A total of 49 AEs were reported during this study by 20 subjects. Four subjects did not report any adverse event. A total of 18 AEs (18 of 49; 37%) were judged to be possibly or probably related to study treatment, with 2 of these AEs reported pre-dose. Fifteen of all reported AEs (15 of 49 events; 31%) occurred pre-dose and were reported by 10 subjects.

Immunogenicity Results: Sera tested at predose, Day 18, and Day 32 from all 24 subjects had no detectable anti-hPTH (1-34) antibodies.

### Example 8

A phase 1, open-label, randomized, crossover study of Macroflux hPTH patch and teriparatide PTH (Forteo™) was conducted in thirty four healthy postmenopausal women. The purpose of the study was to determine the dose and application site combination of Macroflux hPTH that is most comparable to FORTEO 20 μg injected subcutaneously (SC) to the abdomen. Additionally, tolerability and the topical and systemic safety of Macroflux hPTH were also evaluated. The subjects were treated once per day, on four consecutive days in a randomized fashion with Macroflux hPTH consisting of 30 μg on the abdomen, 40 μg on the abdomen, or 40 μg on the thigh, or FORTEO 20 μg SC abdomen as control. All microprojection arrays were applied with 0.20 J/cm² of force and were left in place for 30 minutes. The Macroflux microprojection arrays have microprojection length of 200 μm and a surface area of 2 cm² with 725 microprojections/cm².

To compare the pharmacokinetics of the three application methods of Macroflux® hPTH (1-34) to SC FORTEO®, AUC and Cₘₐₓ were calculated. Plasma concentrations of hPTH were measured in blood samples collected at 0 (predose), 5, 10, 15, and 30 minutes, 1, 2, 3, 4, and 8 hours after dosing initiation. Plasma concentrations of hPTH were measured in blood samples collected at 0 (predose), 5, 10, 15, and 30 minutes, 1, 2, 3, 4, and 8 hours after dosing initiation.

Plasma hPTH (1-34) concentration as a function of time following Macroflux® HPTH was plotted and compared to that of SC FORTEO® HPTH (1-34). The following pharmacokinetic parameters including AUC_{inf}, Cₘₐₓ, Tₘₐₓ, and t_{1/2} were calculated for each treatment and by subject. In addition, dose-normalized AUC and Cₘₐₓ were calculated.

Serum concentrations of the biomarkers total calcium, ionized calcium, phosphate, albumin, and total protein were measured in blood samples collected at 0 (predose), 1, 2, 3, 4, and 8 hours after dosing initiation.

Serum concentrations of total and ionized calcium, corrected calcium, phosphate, albumin, and total protein were obtained at each measured time point for all treatment groups and descriptive statistics presented.

Descriptive statistics were presented for urinary concentrations of cAMP and phosphate, each adjusted for creatinine concentration, at each measured time point for all treatments. Cyclic AMP and phosphate measurements were presented as a ratio to creatinine. Mean changes from baseline were calculated for each parameter and compared between treatment groups.

TABLE 21
Mean ± SD Values for Plasma hPTH Pharmacokinetic Parameters

| | Treatment | | | |
|---|---|---|---|---|
| | Mean ± SD, n = 34 | | | |
| | Macroflux ® hPTH (1-34) | | | SC FORTEO ® |
| | Abdomen | Abdomen | Thigh | Abdomen |
| Parameter | 30 μg | 40 μg | 40 μg | 20 μg |
| Cₘₐₓ (pg/mL) | 133.5 ± 58.2 | 133.7 ± 63.9 | 85.6 ± 52.4 | 107.3 ± 39.8 |
| Tₘₐₓ (h) | 0.13 ± 0.05 | 0.11 ± 0.04 ^{a} | 0.14 ± 0.09 ^{b} | 0.55 ± 0.24 |
| t_{1/2} (h) | 0.82 ± 0.32 ^{c} | 0.71 ± 0.24 ^{d} | 0.95 ± 0.59 ^{e} | 1.13 ± 0.60 ^{f} |
| AUCₜ (pg · h/mL) | 78.3 ± 44.8 | 80.0 ± 54.4 ^{a} | 57.3 ± 50.4 ^{b} | 130.9 ± 50.3 |
| CV (%) | 57.2 | 68.0 | 88.0 | 38.4 |
| AUC₍₀₋₈₎ (pg · h/mL) | 89.2 ± 43.4 | 90.5 ± 54.2 ^{a} | 65.6 ± 50.7 ^{b} | 144.7 ± 53.6 |
| CV (%) | 48.7 | 59.9 | 77.3 | 37.0 |
| AUC_{inf} (pg · h/mL) | 103.9 ± 39.6 | 103.5 ± 52.6 ^{a} | 80.7 ± 52.5 ^{b} | 170.8 ± 54.9 |
| CV (%) | 38.1 | 50.9 | 65.0 | 32.1 |
| Relative exposure g (%) | 68.6 ± 42.2 | 64.0 ± 33.5 | 57.1 ± 64.9 | REFERENCE |

^{a} n = 33;
^{b} n = 32;
^{c} n = 29,
^{d} n = 25,
^{e} n = 20,
^{f} n = 6
^{g} % AUC values relative to that of FORTEO.

The 30 μg and 40 μg Macroflux® HPTH applications to the abdomen achieved similar mean Cₘₐₓ and AUC values, which were approximately 64-69% relative exposure (AUC) with 25% higher Cₘₐₓ compared to SC FORTEO® 20 μg injection in the abdomen. See Table 21. There were, however, differences between the two application sites (abdomen and thigh) with 40 μg Macroflux® hPTH application to the thigh generally resulted in mean Cₘₐₓ and AUC values 36% and up to 25% lower, respectively, than that for application to the abdomen (30 and 40 μg).

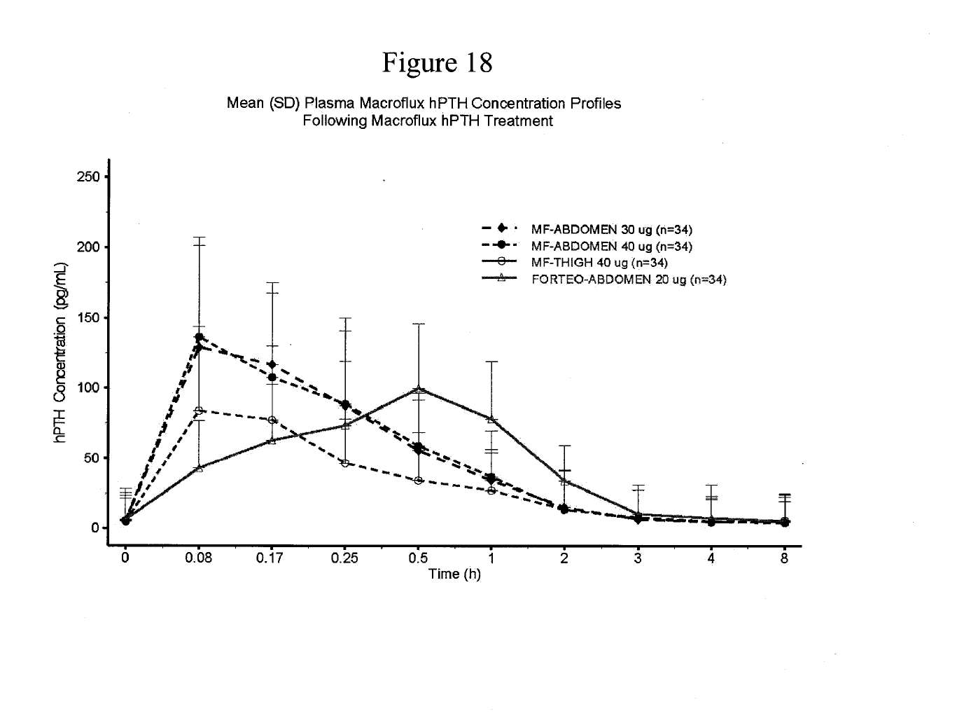

As indicated in the mean concentration profile, the time to reach peak concentration of teriparatide was faster with Macroflux® hPTH than with FORTEO®, as demonstrated by relatively shorter mean Tₘₐₓ value (0.11 to 0.14 hour versus 0.55 hour, respectively, p<0.0001). See FIG. 18 and Table 21. The mean terminal half-life for teriparatide was somewhat shorter with Macroflux® hPTH application to the abdomen and to the thigh (0.71 to 0.95 hours) compared to that for FORTEO® (1. 13 hours). The half-life was only determined for 6 (6/34; 17.6%) subjects after treatment with FORTEO® as compared to between 20 and 29 subjects (58.8% to 85.3%) after the Macroflux® hPTH applications, so this may explain the difference in the half-life between the treatments. The two application sites for Macroflux® hPTH had similar mean Tₘₐₓ and terminal half-life values.

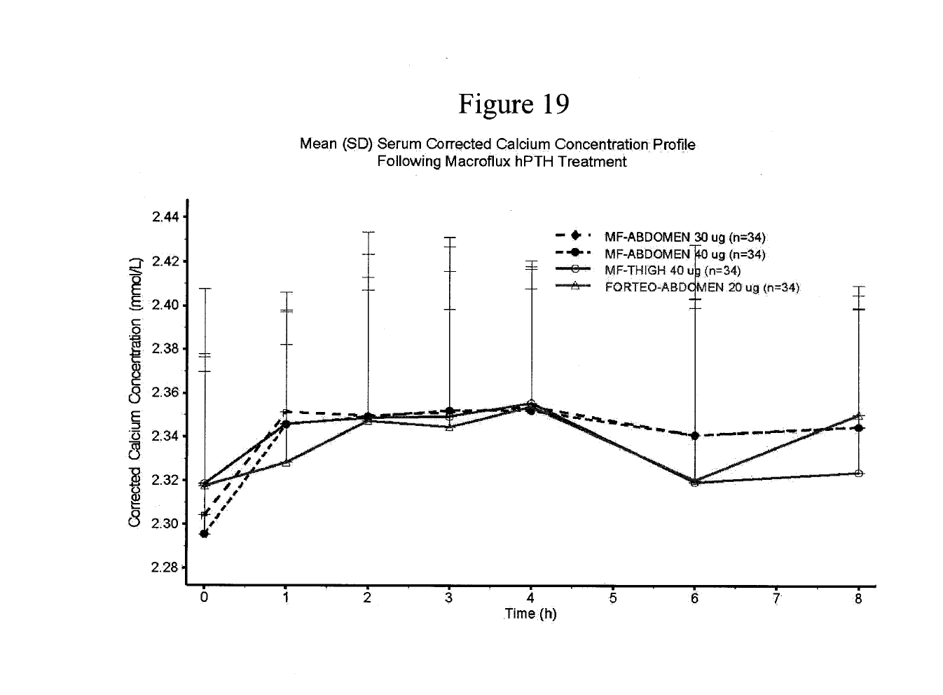

All the observed pharmacodynamic changes were consistent with the known pharmacologic effects of HPTH. Similar to SC FORTEO®, Macroflux® hPTH treatment resulted in significant changes (p<0.05) in all hPTH-responsive biomarkers. Both SC FORTEO® and Macroflux® hPTH treatments led to similar small but significant increased concentrations of serum total, corrected, and ionized calcium (p<0.05), and adjusted urinary cAMP excretion, as would be expected for pharmacodynamic effects of hPTH (p<0.0001). Both Macroflux® hPTH and FORTEO~treatments showed significantly increased concentrations of corrected serum calcium above baseline at 4 hours compared to pretreatment (p<0.001) with at 0.085±0.062 (30 μg abdomen), 0.080±0.098 (40 μg abdomen) and 0.075+0.052 mmol/L (40 μg thigh) for Macroflux and 0.070+0.053 mmol/L for 20 μg SC FORTEO. See FIG. 19.

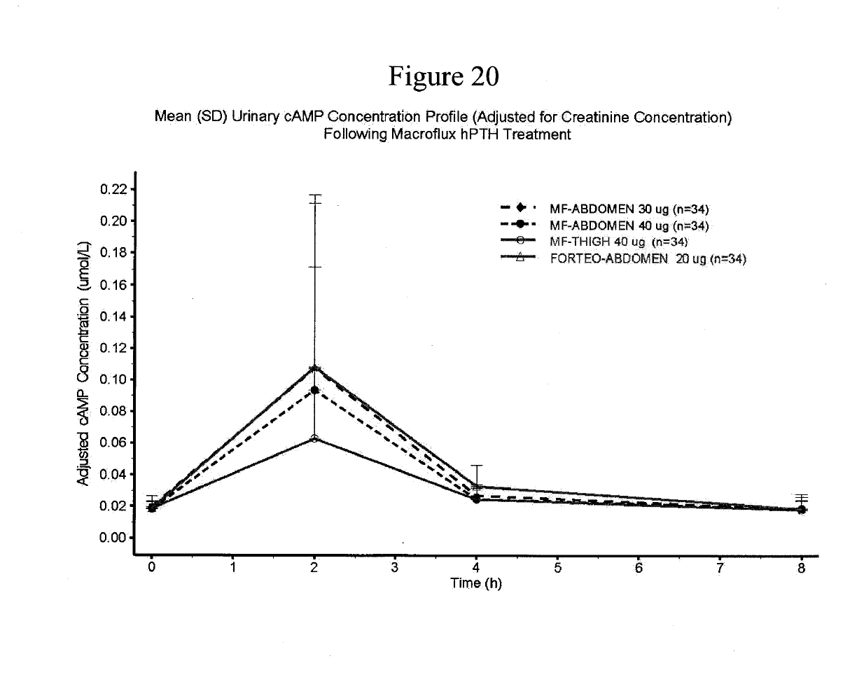

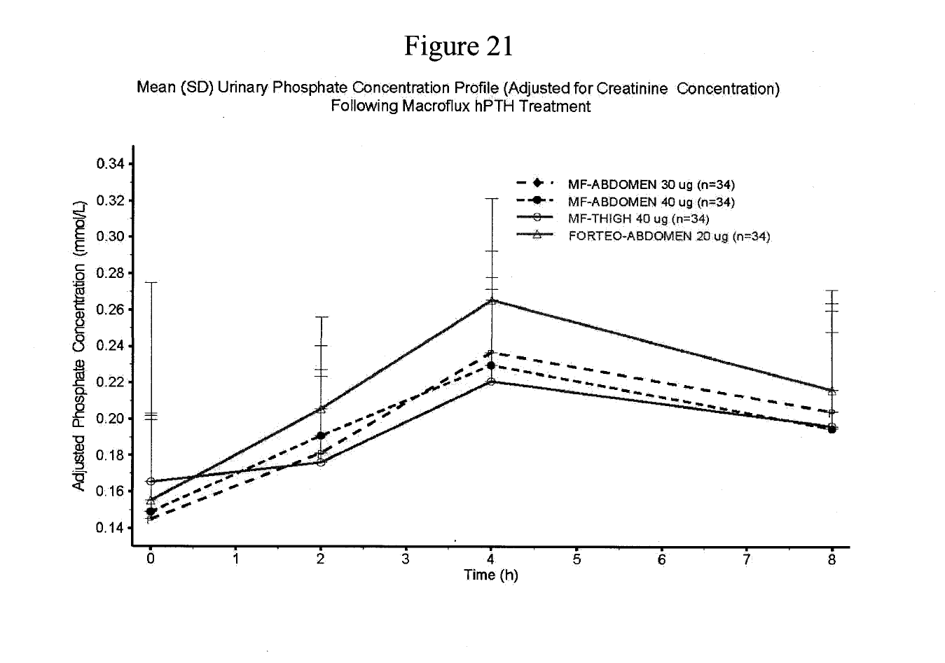

Both SC FORTEO® and Macroflux® hPTH treatments were similar in showing reduced concentrations of serum phosphate, as would be expected for pharmacodynamic effects of hPTH (p<0.001). Both SC FORTEO® and Macroflux® hPTH treatments showed significantly increased concentrations of adjusted urinary cAMP (p<0.0001) in pooled urine samples at 2 hours (0-2 hours) compared to pre-dose levels. See FIG. 20. In addition, adjusted urinary phosphate concentrations increased significantly from pre-dose levels after SC FORTEO® and Macroflux® hPTH treatments (p<0.0001) in the urine samples pooled at 2-4 hours. See FIG. 21. No treatment differences were seen in the change from pre-dose concentrations of serum albumin and total protein.

No serious adverse events (SAEs) were reported, and no subject discontinued from the study because of an adverse event (AE). Macroflux® hPTH was well-tolerated. A total of 50 AEs were reported during this study in 25 subjects who received Macroflux® hPTH application (27 AEs after application to 16 subjects) or SC FORTEO® (16 AEs after injection of 9 subjects). Nine subjects did not report any AE. Seven of the reported AEs (7 of 50 events; 14%) occurred pre-dose and were reported by 5 subjects.

No clinically significant changes were observed for vital signs, clinical laboratory test results, physical examination findings, and ECG results during the study.

As will be appreciated by one having ordinary skill in the art, the present invention provides numerous advantages. For example, a microprojection based apparatus and method has the advantage of transdermal delivery of a PTH-based agent exhibiting a PTH-based agent pharmacokinetic profile similar to that observed following subcutaneous administration. Another advantage is transdermal delivery of a PTH-based agent with rapid on-set of biological action. Yet another advantage is transdermal delivery of a PTH-based agent with sustained biological action for a period of up to 8 hours. Further, transdermal delivery from a microprojection array coated with a 10- 100 μg dose of teriparatide (hPTH. (1-34)) results in a plasma Cₘₐₓ of at least 50 μg/mL after one application.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for preventing or treating osteopenia, comprising the steps of:
providing a transdermal delivery device having disposed thereon at least one hPTH-based formulation;
applying said transdermal device to a skin site of said patient to deliver hPTH to said patient; wherein said formulation achieves a mean Cmax value when applied to the thigh of said patient that is about 15% to about 75% of a mean Cmax value achieved by said formulation when applied to the abdomen of said patient.

2. The method of claim 1 wherein said formulation achieves a mean Cmax value when applied to the thigh of said patient that is about 20% to about 60% of a mean Cmax value achieved by said formulation when applied to the abdomen of said patient.

3. The method of claim 1 wherein said formulation achieves a mean Cmax value when applied to the thigh of said patient that is about 25% to about 35% of a mean Cmax value achieved by said formulation when applied to the abdomen of said patient.

4. The method of claim 1 wherein said formulation achieves a mean plasma hPTH Tmax of 5 minutes or less.

5. The method of claim 1, wherein said method comprises achieving a HPTH mean plasma Cmax value of at least 50 pg/mL.

6. The method of claim 1, wherein said method comprises achieving a hPTH mean plasma Cmax value of at least 100 pg/mL.

7. The method of claim 1, wherein after 3 hours from applying said transdermal device to the patient's skin, said method achieves a hPTH plasma concentration of no more than about 10 pg/mL.

8. The method of claim 1, wherein after 2 hours from applying said transdermal device to the patient's skin, said method achieves a hPTH plasma concentration of no more than about 20 pg/mL.

9. The method of claim 1, wherein after 1 hour from applying said transdermal device to the patient's skin, said method achieves a hPTH plasma concentration of no more than about 30 pg/mL.

10. The method of claim 1, wherein the ratio between the Tmax achieved by said method and the Tmax achieved by subcutaneous administration of said hPTH-based agent is from about 1:2 to about 1:10.

11. The method of claim 1, wherein said device is applied to the abdomen of said patient and the ratio between the Tmax achieved by said method and the Tmax achieved by subcutaneous administration of said hPTH-based agent is from about 1:4 to about 1:6.

12. The method of claim 1, wherein said device is applied to the skin of said patient for a period of about 30 minutes and the residual hPTH remaining on said device after application is about 40% to about 75% of hPTH present on said device prior to application of said device to the skin of said patient.

13. The method of claim 1, wherein said skin site of said patient is on the abdomen of said patient.

14. The method of claim 1, wherein said formulation comprises a hPTH-based agent selected from the group consisting of hPTH (1-34), hPTH salts and analogs, teriparatide and related peptides.

15. The method of claim 14, wherein said hPTH salt is selected from group consisting of acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

16. The method of claim 1, wherein said formulation comprises teriparatide (hPTH (1-34)) in the range of approximately 10-100 μg.

17. The method of claim 1, wherein the method prevents or delays onset of osteoporosis.

18. The method of claim 1, wherein the method prevents or delays the onset of osteoporotic fractures.

19. The method of claim 1, wherein the method reduces severity of osteoperosis deleterious effects.

20. The method of claim 1, wherein the method reduces severity of osteoporotic fractures.

21. A method for preventing or treating osteopenia, comprising the steps of:
providing a microprojection member having a plurality of stratum corneum-piercing microprotrusions; said microprojection member having a coating disposed thereon, said coating including at least one hPTH-based formulation;
applying said microprojection member to a skin site of said patient, whereby said plurality of stratum corneum-piercing microprotrusions pierce the stratum corneum and deliver hPTH to said patient;
removing said microprojection member from said skin site;
wherein said formulation achieves a mean Cmax value when applied to the thigh of said patient that is about 15% to about 75% of a mean Cmax value achieved by said formulation when applied to the abdomen of said patient.

22. The method of claim 21 wherein said formulation achieves a mean Cmax value when applied to the thigh of said patient that is about 20% to about 60% of a mean Cmax value achieved by said formulation when applied to the abdomen of said patient.

23. The method of claim 21 wherein said formulation achieves a mean Cmax value when applied to the thigh of said patient that is about 25% to about 35% of a mean Cmax value achieved by said formulation when applied to the abdomen of said patient.

24. The method of claim 21 wherein said formulation achieves a mean plasma hPTH Tmax of 5 minutes or less.

25. The method of claim 21, wherein said method comprises achieving a hPTH mean plasma Cmax value of at least 50 pg/mL.

26. The method of claim 21, wherein said method comprises achieving a hPTH mean plasma Cmax value of at least 100 pg/mL.

27. The method of claim 21, wherein after 3 hours from applying said transdermal device to the patient's skin, said method achieves a HPTH plasma concentration of no more than about 10 pg/mL.

28. The method of claim 21, wherein after 2 hours from applying said transdermal device to the patient's skin, said method achieves a HPTH plasma concentration of no more than about 20 pg/mL.

29. The method of claim 21, wherein after 1 hour from applying said transdermal device to the patient's skin, said method achieves a hPTH plasma concentration of no more than about 30 pg/mL.

30. The method of claim 21, wherein the ratio between the Tmax achieved by said method and the Tmax achieved by subcutaneous administration of said hPTH-based agent is from about 1:2 to about 1:10.

31. The method of claim 21, wherein said device is applied to the abdomen of said patient and the ratio between the Tmax achieved by said method and the Tmax achieved by subcutaneous administration of said hPTH-based agent is from about 1:4 to about 1:6.

32. The method of claim 21, wherein said device is applied to the skin of said patient for a period of about 30 minutes and the residual HPTH remaining on said device after application is about 40% to about 75% of hPTH present on said device prior to application of said device to the skin of said patient.

33. The method of claim 21, wherein said skin site of said patient is on the abdomen of said patient.

34. The method of claim 21, wherein said formulation comprises a hPTH-based agent selected from the group consisting of HPTH (1-34), hPTH salts and analogs, teriparatide and related peptides.

35. The method of claim 34, wherein said hPTH salt is selected from group consisting of acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

36. The method of claim 21, wherein said formulation comprises teriparatide (HPTH (1-34)) in the range of approximately 10-100 μg.

37. The method of claim 21, wherein said formulation comprises teriparatide (hPTH (1-34)) in a dose of approximately 10 μg.

38. The method of claim 21, wherein said formulation comprises teriparatide (hPTH (1-34)) in a dose of approximately 20 μg.

39. The method of claim 21, wherein said formulation comprises teriparatide (hPTH (1-34)) in a dose of approximately 30 μg.

40. The method of claim 21, wherein said formulation comprises teriparatide (HPTH (1-34)) in a dose of approximately 40 μg.

41. The method of claim 21, wherein the method prevents or delays onset of osteoporosis.

42. The method of claim 21, wherein the method prevents or delays the onset of osteoporotic fractures.

43. The method of claim 21, wherein the method reduces severity of osteoperosis deleterious effects.

44. The method of claim 21, wherein the method reduces severity of osteoporotic fractures.

45. A method for preventing or treating osteopenia, comprising the steps of:
providing a transdermal delivery device having disposed thereon at least one hPTH-based formulation;
applying said transdermal device to a skin site located on the abdomen of said patient to deliver hPTH to said patient; wherein said formulation achieves a mean tmax value of 30 minutes or less.

46. The method of claim 45, wherein said formulation achieves a mean tmax value of 20 minutes or less.

47. The method of claim 45, wherein said formulation achieves a mean tmax value of 10 minutes or less.

48. The method of claim 45, wherein said formulation achieves a mean tmax value of 5 minutes or less.

49. A method for preventing or treating osteopenia, comprising the steps of:
providing a transdermal delivery device having disposed thereon at least one hPTH-based formulation;
applying said transdermal device to a skin site located on the thigh of said patient to deliver HPTH to said patient; wherein said formulation achieves a mean tmax value of 30 minutes or less.

50. The method of claim 49, wherein said formulation achieves a mean tmax value of 20 minutes or less.

51. The method of claim 49, wherein said formulation achieves a mean tmax value of 10 minutes or less.

52. The method of claim 49, wherein said formulation achieves a mean tmax value of 5 minutes or less

53. A method for preventing or treating osteopenia, comprising the steps of:
providing a microprojection member having a plurality of stratum corneum-piercing microprotrusions; said microprojection member having a coating disposed thereon, said coating including at least one hPTH-based formulation;
applying said microprojection member to a skin site located on the abdomen of said patient, wherein said formulation achieves a mean tmax value of 30 minutes or less.

54. The method of claim 53, wherein said formulation achieves a mean tmax value of 20 minutes or less.

55. The method of claim 53, wherein said formulation achieves a mean tmax value of 10 minutes or less.

56. The method of claim 53, wherein said formulation achieves a mean tmax value of 5 minutes or less.

57. A method for preventing or treating osteopenia, comprising the steps of:
providing a microprojection member having a plurality of stratum corneum-piercing microprotrusions; said microprojection member having a coating disposed thereon, said coating including at least one hPTH-based formulation;
applying said microprojection member to a skin site located on the thigh of said patient, wherein said formulation achieves a mean tmax value of 30 minutes or less.

58. The method of claim 57, wherein said formulation achieves a mean tmax value of 20 minutes or less.

59. The method of claim 57, wherein said formulation achieves a mean tmax value of 10 minutes or less.

60. The method of claim 57, wherein said formulation achieves a mean tmax value of 5 minutes or less.

61. A method for preventing or treating osteopenia, comprising the steps of:
providing a transdermal delivery device having disposed thereon at least one hPTH-based formulation comprising teriparatide (hPTH (1-34)) in a dose of approximately 40 μg;
applying said transdermal device to a skin site of said patient to deliver hPTH to said patient; wherein said formulation achieves a mean tmax value of 30 minutes or less.

62. The method of claim 61, wherein said formulation achieves a mean tmax value of 20 minutes or less.

63. The method of claim 61, wherein said formulation achieves a mean tmax value of 10 minutes or less.

64. The method of claim 61, wherein said formulation achieves a mean tmax value of 5 minutes or less.

65. A method for preventing or treating osteopenia, comprising the steps of:
providing a transdermal delivery device having disposed thereon at least one hPTH-based formulation comprising teriparatide (hPTH (1-34)) in a dose of approximately 40 μg;
applying said transdermal device to a skin site of said patient to deliver HPTH to said patient; wherein said formulation achieves a mean tmax value of 30 minutes or less.

66. The method of claim 65, wherein said formulation achieves a mean tmax value of 20 minutes or less.

67. The method of claim 65, wherein said formulation achieves a mean tmax value of 10 minutes or less.

68. The method of claim 65, wherein said formulation achieves a mean tmax value of 5 minutes or less

69. A method for preventing or treating osteopenia, comprising the steps of:
providing a microprojection member having a plurality of stratum corneum-piercing microprotrusions; said microprojection member having a coating disposed thereon, said coating including at least one hPTH-based formulation comprising teriparatide (hPTH (1-34)) in a dose of approximately 40 μg;
applying said microprojection member to a skin site of said patient, wherein said formulation achieves a mean tmax value of 30 minutes or less.

70. The method of claim 69, wherein said formulation achieves a mean tmax value of 20 minutes or less.

71. The method of claim 69, wherein said formulation achieves a mean tmax value of 10 minutes or less.

72. The method of claim 69, wherein said formulation achieves a mean tmax value of 5 minutes or less.

73. A method for preventing or treating osteopenia, comprising the steps of:
providing a microprojection member having a plurality of stratum corneum-piercing microprotrusions; said microprojection member having a coating disposed thereon, said coating including at least one hPTH-based formulation comprising teriparatide (hPTH (1-34)) in a dose of approximately 40 μg;
applying said microprojection member to a skin site of said patient, wherein said formulation achieves a mean tmax value of 30 minutes or less.

74. The method of claim 73, wherein said formulation achieves a mean tmax value of 20 minutes or less.

75. The method of claim 73, wherein said formulation achieves a mean tmax value of 10 minutes or less.

76. The method of claim 73, wherein said formulation achieves a mean tmax value of 5 minutes or less.
